(19) United States
(12) Patent Application Publication
(10) Pub. No.: US 2026/0263185 A1
(43) Pub. Date: Sep. 10, 2026

(54) RESTORATION OF A FLEXIBLE ELONGATE DEVICE

(71) Applicant: Intuitive Surgical Operations, Inc., Sunnyvale, CA (US)

(72) Inventors: Samuel B. Schorr, Sunnyvale, CA (US); Shibing Liu, Sunnyvale, CA (US); Federico Barbagli, Sunnyvale, CA (US)

(73) Assignee: Intuitive Surgical Operations, Inc., Sunnyvale, CA (US)

(21) Appl. No.: 19/562,069

(22) Filed: Mar. 10, 2026

Related U.S. Application Data
(60) Provisional application No. 63/769,546, filed on Mar. 10, 2025.

(51) Int. Cl. A61B 34/00; A61B 10/02; A61B 34/30; A61B 90/00

(52) U.S. Cl. CPC A61B 34/76; A61B 10/0233; A61B 34/30; A61B 90/06; A61B 90/37; A61B 2034/301; A61B 2090/066; A61B 2090/3764

(57) ABSTRACT

A medical system includes a manipulator assembly configured to drive a flexible elongate device and a control system coupled to the manipulator assembly. The control system is configured to determine to enter a loss of actuation state for the flexible elongate device. The control system is further configured to determine and store a first physical parameter of an articulable body portion of the flexible elongate device prior to entering the loss of actuation state. The control system is further configured to control the manipulator assembly to enter the loss of actuation state for the flexible elongate device, where the first physical parameter is not maintained in the loss of actuation state. The control system is further configured to determine to exit the loss of actuation state and, in response to determining to exit the loss of actuation state, control the manipulator assembly to restore the first physical parameter.

### FIELD

Disclosed embodiments relate to improved robotic and/or medical (including surgical) devices, systems, and methods.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter or bronchoscope, that can be inserted into anatomic passageways and navigated toward a region of interest within the patient anatomy. For a variety of reasons, the flexible elongate device may enter a loss of actuation state, where one or more physical parameters of the flexible elongate device (e.g., a bending angle of its distal end) is not maintained. Accordingly, there exists a need to restore the one or more physical parameters of the flexible elongate device in response to exiting, or determining an exit from, the loss of actuation state.

### SUMMARY

The following presents a simplified summary of various examples described herein and is not intended to identify key or critical elements or to delineate the scope of the claims.

In general, in one aspect, one or more embodiments of the disclosure relate to a medical system including a manipulator assembly configured to drive a flexible elongate device and a control system coupled to the manipulator assembly. The control system is configured to determine to enter a loss of actuation state for the flexible elongate device, where the loss of actuation state can include one of a loss of pose state or a loss of apposition state. The control system is further configured to determine and store a first physical parameter of an articulable body portion of the flexible elongate device prior to entering the loss of actuation state. The control system is further configured to control the manipulator assembly to enter the loss of actuation state for the flexible elongate device, where the first physical parameter is not maintained for the articulable body portion by the manipulator assembly in the loss of actuation state. The control system is further configured to determine to exit the loss of actuation state for the flexible elongate device and, in response to determining to exit the loss of actuation state, control the manipulator assembly to restore the first physical parameter for the articulable body portion of the flexible elongate device.

In general, in one aspect, one or more embodiments of this disclosure relate to a method for controlling a medical system. The medical system includes a manipulator assembly configured to drive a flexible elongate device. The method is performed by a control system of the medical system. The method includes determining to enter a loss of actuation state for the flexible elongate device, where the loss of actuation state can include one of a loss of pose state or a loss of apposition state. The method further includes determining and storing a first physical parameter of an articulable body portion of the flexible elongate device prior to entering the loss of actuation state. The method further includes controlling the manipulator assembly to enter the loss of actuation state for the flexible elongate device, where the first physical parameter is not maintained for the articulable body portion by the manipulator assembly in the loss of actuation state. The method further includes determining to exit the loss of actuation state for the flexible elongate device and, in response to determining to exit the loss of actuation state, controlling the manipulator assembly to restore the first physical parameter for the articulable body portion of the flexible elongate device.

In general, in one aspect, one or more embodiments of this disclosure relate to a non-transitory machine-readable medium including a plurality of machine-readable instructions executed by one or more processors associated with a medical system. The medical system includes a manipulator assembly configured to drive a flexible elongate device. The plurality of machine-readable instructions causing the one or more processors to perform a method. The method includes determining to enter a loss of actuation state for the flexible elongate device, where the loss of actuation state may include one of a loss of pose state or a loss of apposition state. The method further includes determining and storing a first physical parameter of an articulable body portion of the flexible elongate device prior to entering the loss of actuation state. The method further includes controlling the manipulator assembly to enter the loss of actuation state for the flexible elongate device, where the first physical parameter is not maintained for the articulable body portion by the manipulator assembly in the loss of actuation state. The method further includes determining to exit the loss of actuation state for the flexible elongate device and, in response to determining to exit the loss of actuation state, controlling the manipulator assembly to restore the first physical parameter for the articulable body portion of the flexible elongate device.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient space including a medical instrument mounted on an insertion assembly according to some embodiments.

FIG. 4 is a simplified diagram of a medical system including an imaging device in accordance with one or more embodiments.

FIG. 5 depicts a flowchart to restore a first physical parameter of a flexible elongate device in accordance with one or more embodiments.

FIGS. 6A, 6B, 6C, 6D, and 6E depict various views of an anatomical structure, a flexible elongate device, and a relationship between the anatomical structure and the elongate device, prior to, during, and after a loss of actuation state, according to some embodiments.

FIG. 7 depicts a flowchart to restore a physical parameter of a flexible elongate device having entered and exited a loss of actuation state in accordance with one or more embodiments.

FIG. 8 depicts a flowchart to restore a physical parameter of a flexible elongate device in response to a user causing the flexible elongate device to enter and exit a loss of actuation state in accordance with one or more embodiments.

FIG. 9 is a flowchart of a method according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Embodiments of the disclosure include medical systems and methods for operating such medical systems. A medical system that uses flexible elongate devices (e.g., catheters, bronchoscopes, endoscopes, etc.) can be used to move a flexible elongate device, or a portion of a flexible elongate device; including a medical tool that may be enclosed by the flexible elongate device. A medical system can be used to perform a medical operation. For example, medical operation such as a biopsy can be performed using a medical system that includes and/or uses flexible elongate devices (e.g., catheters or endoscopes) by inserting a biopsy needle tool through the flexible elongate device at a target site.

Performing a medical operation may involve navigation and localization tasks of the flexible elongate device. Performing a medical operation may also involve positioning a portion of the flexible elongate device so that it, or an associated tool or probe (e.g., an ultrasound sensor), is in surface contact with an adjacent anatomical surface, where this surface contact may be described as apposition. Thus, performing a medical operation may also involve monitoring, and/or visualizing, other actions. The task of navigation can include following a passage such as an airway or other anatomical passageway. Further, navigation (or navigating) may refer to the movement of the flexible elongate device to a target region or target. For example, during a diagnostic or therapeutic procedure such as bronchoscopy, a flexible elongate device may be inserted through a naturally or surgically created anatomic orifice of a patient (e.g., nose, mouth, tracheostomy) through the tracheobronchial tree to a target region (e.g., region containing or proximate to a peripheral pulmonary lesion (PPL) designated as a target). Navigating can include articulation of the flexible elongate device at any portion along the length of the flexible elongate device. Further, navigating can be associated with bulk movement of the flexible elongate device relative to a so-called insertion axis of the medical system controlling the movement of the flexible elongate device. That is, navigation may involve significant movement along an insertion degree of freedom, in addition to articulation of one or more articulable portions (e.g., distal portion) of the flexible elongate device using one or more articulation degrees of freedom.

Localization generally refers to articulation of the distal portion of the flexible elongate device to aim (or point or direct) the distal end of the flexible elongate device at a specified target or target structure in the target region. Thus, localization can include a targeting operation (or “aiming” operation) and other such pre-targeting movements performed local to the target region, where the targeting operation includes aiming the flexible elongate device towards the target.

Apposition may involve a portion of the flexible elongate device or a portion of an instrument inserted via the flexible elongate device in surface contact with an adjacent anatomical surface.

Herein, the term “movement” can be used to describe navigation, localization, or apposition tasks or operations. In some implementations, apposition may be performed as a navigation or localization task. That is, movement along an insertion degree of freedom and/or articulation of one or more articulable portions of the flexible elongate device—whether used for navigation, localization, or apposition—can be described as movement of the flexible elongate device. Embodiments of this disclosure are applicable to navigating, targeting, and apposition. As such, changes in a flexible elongate device from a first position to a second position (or, more generally from a first pose or shape to a second pose or shape) are described as “movements” (including other references such as “move” and “to move”). Further, a movement may be composed of an ordered sequence of movements. For example, a flexible elongate device can be moved from a first shape to a second shape while passing through any number of intermediate shapes, where changes between intermediate shapes can be described as movements.

Use of the flexible elongate device, e.g., in navigation and localization tasks including a targeting operation, involves articulation of one or more articulable portions of the flexible elongate device. One or more physical parameters can correspond to the flexible elongate device. These physical parameters can include, but are not limited to: a position and/or orientation of the flexible elongate device or portion thereof; a bend angle of a portion of the flexible elongate device; an orientation of an axis colinear or tangent to a portion of the flexible elongate device; and a distance from a target relative to the distal end of the flexible elongate device. For various reasons described below, during operation of the medical system, a flexible elongate device may enter a “loss of actuation state.” In the loss of actuation state, the one or more physical parameters corresponding to the flexible elongate device is not maintained. For example, during a targeting operation (i.e., localization), an articulable portion of the flexible elongate device can be used to aim (or direct or point) the distal end of the flexible elongate device at a target, where the one or more physical parameters corresponding to the aimed flexible elongate device include a position (e.g., x, y, and z coordinates in a Cartesian space) of the distal end of the flexible elongate device. As another example, once aimed (or completion of a targeting operation), the one or more physical parameters of the aimed flexible elongate device can include an orientation of an axis colinear with a distal portion of the flexible elongate device (e.g., a pointing direction) with respect to the target and/or a distance of the distal end of the flexible elongate device from the target. As yet another example, the aimed flexible elongate device may be in a configuration that involves apposition. The apposition may involve a portion of the flexible elongate device or a portion of an instrument inserted via the flexible elongate device in surface contact with an adjacent anatomical surface. The one or more physical parameters that enable or describe apposition may be, for example, positional and/or orientational degrees of freedom of the flexible elongate device. While apposition/loss of apposition may be characterized using the previously discussed positional/orientational variables, apposition/loss of apposition may also be characterized using different or additional physical parameters such as a binary apposition yes/no characterization or an amount of force present during the apposition (e.g., measured using a force sensor), the location and/or direction when in the apposition, etc. In some implementations, the loss of actuation state may further be characterized as a loss of pose state or a loss of apposition state. That is, in some implementations, the loss of actuation state includes, or is one of, a loss of pose state or a loss of apposition state. A loss of pose state may differ from a loss of apposition state in the one or more physical parameters used to describe the flexible elongate device, or portion thereof. For example, the loss of pose state may involve a physical parameter of the pose of the flexible elongate device (or, more specifically, a distal end of the flexible elongate device) and the loss of apposition state may involve a physical parameter of a contact force as sensed by a portion of the flexible elongate device (e.g., the contact force representing a force between the portion of the flexible elongate device and an adjacent anatomical structure). In response to entering the loss of actuation state, the one or more physical parameters is not maintained. For example, the position of the distal end of the flexible elongate axis can deviate, depart, or otherwise change value from its position when considered aimed (or upon completion of the targeting operation). As another example, in the loss of actuation state, the orientation of an axis related to the distal end of the flexible elongate device and/or the distance of its distal end from the target can change (i.e., not maintained) relative to the values of these physical parameters while the flexible elongate device was considered aimed. As yet another example, in the loss of actuation state, an apposition may be detected as no longer being present, being present with a reduced apposition force, an altered apposition position/direction, etc.

A more detailed discussion of the medical system, a medical instrument including a flexible elongate device, and methods relating to the restoration of the one or more physical parameters of the flexible elongate device in response to entering and exiting the loss of actuation state, including resulting benefits, is provided below in reference to the figures. In particular, the methods include, for the flexible elongate device, determining an entrance into the loss of actuation state, determining an exit from the loss of actuation state, and restoring the one or more physical parameters to its value(s) prior to entering the loss of actuation state in response to the determined exit from the loss of actuation state.

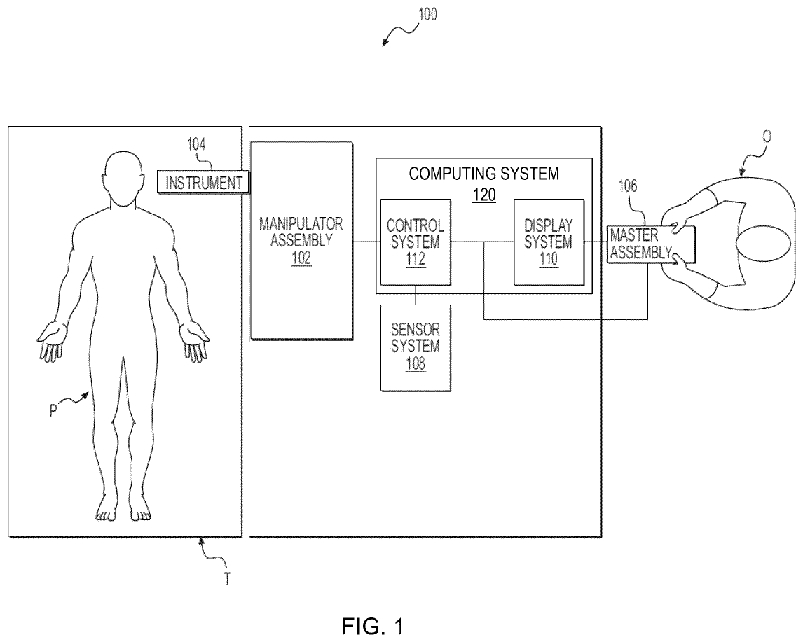

Turning to the figures, FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 (or “leader” assembly) allows an operator O (e.g., a surgeon, a clinician, a physician, or other user) to control the manipulator assembly 102 (or “follower” assembly). In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104. In some examples, the manipulator assembly 102 can be provided on, or as, a movable cart. Thus, the movable cart can be positioned near the table T and patient P as desired by the operator O or their assistants. The movable cart includes a braking system with one or more brakes and a brake sensor. Thus, the moveable cart can be made stationary through activation of the one or more brakes. In one or more implementations, activation of the one or more brakes is performed manually. In other implementations, activation of the one or more brakes is controlled in response to commands, for example, from the control system 112 (e.g., based on an input at the master assembly 106), through user-interaction with a control interface provided at the manipulator assembly 102 itself (e.g., a button or graphical user interface provided at the manipulator assembly 102), or combinations thereof. The brake sensor indicates a state (e.g., “on” or “off,” “active” or “inactive”) and/or quantitative measure of the one or more brakes (e.g., an applied breaking force, a normalized breaking force such as a range from 0% to 100%). As listed below, the brake sensor can be in included in a sensor system 108 of the medical system 100.

The medical system 100 may include a sensor system 108 with one or more sub-systems for receiving information about the manipulator assembly 102 and/or the medical instrument 104. Such sub-systems may include a cart brake sensor (i.e., a brake sensor for the manipulator assembly 102 in instances where the manipulator assembly 102 is provided with, or as, a movable cart); a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models. In some instances, and as described below, intra-operative images can be used to locate and map (or track) the location of the medical instrument 104 as the medical instrument 104 moves through the patient anatomy.

In some examples, for purposes of image-guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, where different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigating and/or targeting assistance to operator O when controlling the medical instrument 104 during an image-guided medical procedure. In general, navigating and targeting sequences can be visualized using the visualization system based on an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

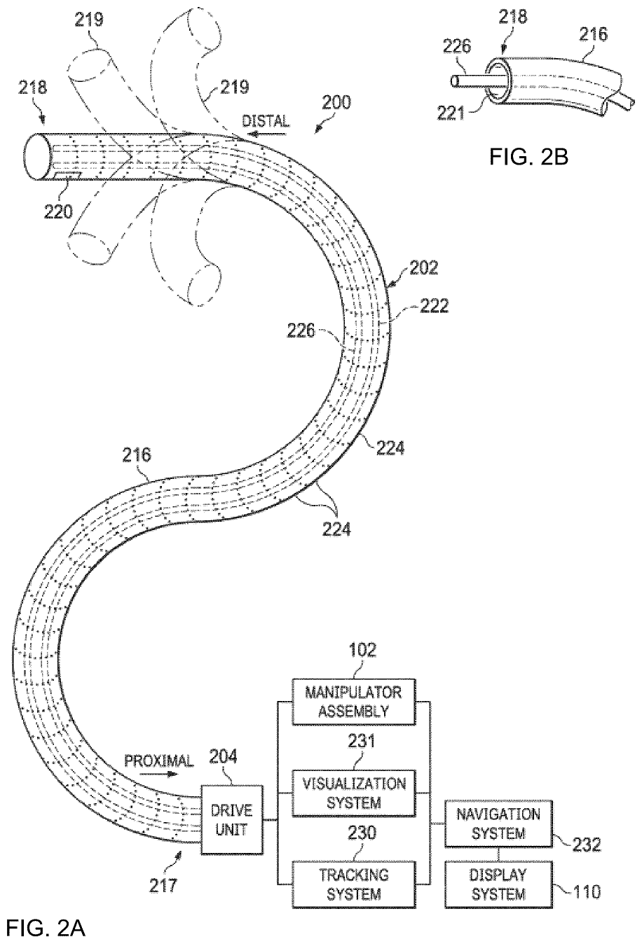

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The flexible elongate device 202 is coupled to the drive unit 204. The flexible elongate device 202 includes a channel 221 through which the medical tool 226 may be inserted. The flexible elongate device 202 navigates within patient anatomy to deliver the medical tool 226 to a procedural site. The flexible elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexible body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of a flexible elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

Embodiments of the instant disclosure use one or more of a shape sensor, a position sensor, a contact force sensor, and intra-operative image data (e.g., macro-level, or externally acquired, images of the anatomy and/or internally acquired images) to determine and store one more physical parameters of the flexible elongate device 202 (or flexible body 216) prior to entering a loss of actuation state. A greater discussion of internally acquired intra-operative image data is provided below. In one or more embodiments, the one or more physical parameters correspond to an articulable body portion of the flexible elongate device 202 such as the distal end 218 of the flexible body 216. In determination of an exit from the loss of actuation state, the manipulator assembly 102 is controlled to restore the one or more physical parameters to the flexible elongate device 202. Restoration of the one or more physical parameters can be guided by (e.g., determination of a trajectory) and/or validated using position, orientation, pose, contact force, and/or shape information obtained using one or more of a shape sensor, position sensor, contact force sensor, and intra-operative image data. A detailed description is provided below.

FIG. 2B is a simplified diagram of the medical tool 226 within the flexible elongate device 202 according to some embodiments. The flexible body 216 of the flexible elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the flexible elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the flexible elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 216 of the flexible elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the flexible elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the flexible elongate device 202 and/or medical tool 226 may include gripping features, manual actuators, or other components for manually controlling the motion of the elongate device 202 and/or medical tool 226. The flexible elongate device 202 may be steerable or, alternatively, the flexible elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the flexible elongate device 202.

In some examples, the medical instrument system 200 (e.g., the flexible elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

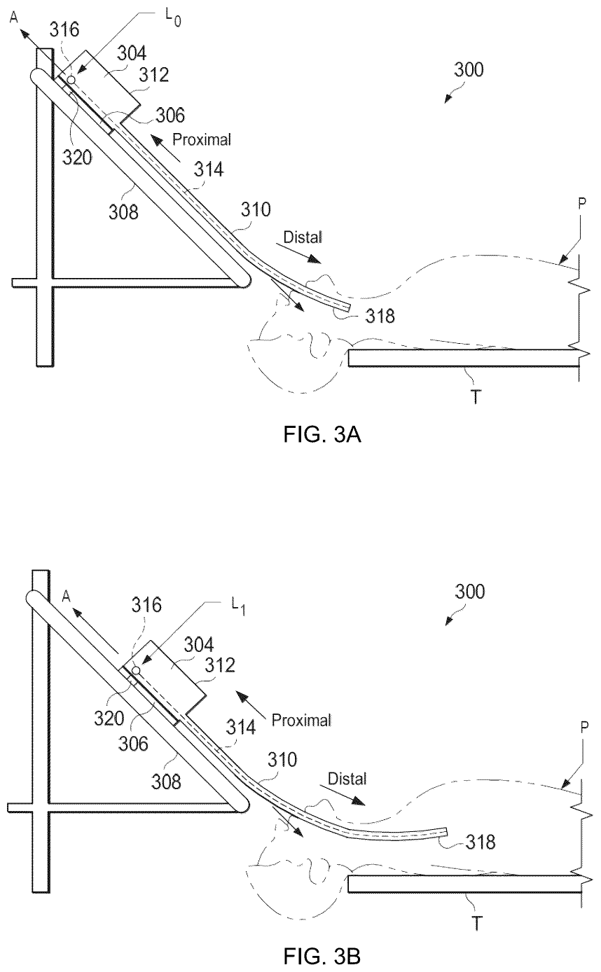

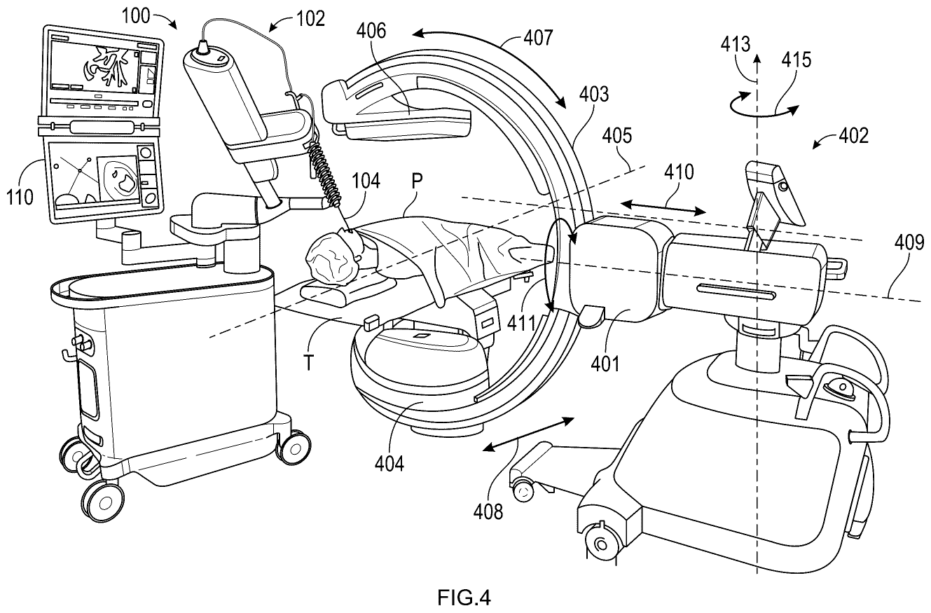

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include a flexible elongate device 310 (e.g., a catheter) coupled to an instrument body 312. Flexible elongate device 310 includes one or more channels sized and shaped to receive a medical tool.

The flexible elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the flexible elongate device 310. The shape sensor 314 may be aligned with the flexible elongate device 310 (e.g., provided within an interior channel or mounted externally). In some examples, the shape sensor 314 may optical fibers used to generate shape information for the flexible elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

The flexible elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the flexible elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the flexible elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the flexible elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the flexible elongate device 310 is inserted into the passageway(s) of the anatomy of patient P.

FIG. 4 depicts another view of a medical system 100, where the medical system 100 is used in conjunction with an imaging system 402. In one or more embodiments, the imaging system is controlled, at least in part, using the medical system (e.g., control system 112). In other embodiments, the medical system receives data (e.g., image data, virtual model, etc.) from the imaging system 402 for various tasks such as visualization and tracking, as previously discussed. As discussed with reference to FIG. 1, the medical system 100 may include a manipulator assembly 102 that controls the operation of a medical instrument 104 in performing various procedures on a patient P. In FIG. 4, patient P is on a table T. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

In accordance with some embodiments, the imaging system 402, which may include more than one imaging device, is capable of acquiring pre- and intra-operative image data, e.g., to construct a virtual model of patient anatomy, visualize a target region, and track, register, and display a location and orientation of a medical instrument (e.g., flexible elongate device) relative to patient anatomy. For example, in some embodiments, the imaging system 402 implements one or more of cone beam computed tomography (CBCT) and fluoroscopy to acquire three-dimensional (3D) and two-dimensional (2D) images, respectively. In other examples, two and a half-dimensional (2.5D) images (or scans) e.g., sectional images that are reconstructed into a 3D volume, may be obtained, for example, using tomosynthesis imaging. In such a case, the imaging system 402 can be used to acquire 2.5D and 2D images. The imaging system 402 can use a combination of imaging devices such as a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device for capturing images, or use any of the aforementioned imaging devices in different modes to acquire images (e.g., of the distal end of medical instrument 104) of mixed dimensionalities including at least a 2D image (e.g., 2D and 3D, 2D and 2.5D).

In the example of FIG. 4, the depicted imaging system 402 is a mobile C-arm style device that can implement fluoroscopy and CBCT to acquire 2D and 3D images (or scans), respectively. The depicted imaging system 402, being mobile, can be translated along a floor. FIG. 4 depicts the translational movement of the imaging system 402 using a first translational degreed of freedom (DOF) 408 and a second translational DOF 410, where the first and second translation DOFs 408, 410 are orthogonal to each other and coplanar with the floor. In general, the translational motion of the imaging system 402 with respect to the floor need not be defined using orthogonal degrees of freedom or an orthogonal coordinate system (e.g., Cartesian). Further, the depicted imaging system 402, by nature of its mobility, can be rotated about itself. For example, a vertical axis 413 (i.e., orthogonal to a plane defined by the floor) may be placed at an arbitrary location (e.g., the center of mass of the imaging system 402) to define a first rotational axis 413 about which the imaging system 402, itself, may be rotated over a first rotational DOF 415. While the imaging system 402 depicted in FIG. 4 is mobile, non-mobile imaging systems 402 may be used without limitation. For example, in some implementations, the imaging system 402, or components of the imaging system 402, can be disposed on a rail system to achieve one or more of the aforementioned degrees of freedom of the imaging system 402. For example, the imaging system 402 may be suspended using a ceiling mounted rail system to allow motion of the imaging device along the first translational DOF 408. In some implementations still, the imaging system 402 can include an extendable boom or rotational joint to realize the second translational DOF 410 and the first rotational DOF 415, respectively. In other implementations, the imaging system 402 is fixed or unmovable in the aforementioned degrees of freedom. In some instances, and as described below, one or more degrees of freedom, for example, the first translational degree of freedom 408, is provided using the table T, where the table T moves relative to a fixed imaging system 402.

Continuing with the example of FIG. 4, the depicted imaging system 402 includes a gantry 401 about which a C-arm 403 is suspended or attached. In some implementations, the gantry 401 defines a second rotational axis 409 and includes a rotational joint to provide rotation of the C-arm 403 over a second rotational DOF 411. The C-arm 403 includes an X-ray generation system 404 and an X-ray collection system 406. The X-ray generation system 404 generates an X-ray beam that is passed through the patient P and collected by the X-ray collection system 406. A detailed description of the inner elements of the X-ray generation system 404 and X-ray collection system 406 exceed the scope of this disclosure. However, in general, an X-ray generation system 404 includes an X-ray tube to generate the X-ray beam, one or more filters to tailor the flux of the generated X-ray beam, and a collimator to direct and shape the X-ray beam. Similarly, in general, an X-ray collection system 406 includes an anti-scatter grid, a detector (e.g., flat panel detector, image intensifiers, etc.), and a dosage meter to measure and validate the received flux.

In one or more implementations, the C-arm 403 of the imaging system 402 defines a third rotational axis 405 that is coaxial with the center of a virtual circle that follows the contours of the C-arm. The C-arm 403, or elements disposed on the C-arm 403 such as the X-ray generation system 404 and the X-ray collection system 406, can be rotated about the third rotational axis 405 over a third rotational DOF 407. In the example of FIG. 4, the imaging system 402 is positioned such that the third rotational axis 405 is approximately aligned with the spine of the patient P. In other instances, the imaging system 402 or patient P may be positioned such that the third rotational axis 405 is substantially coplanar with the patient P (e.g., coplanar with the spine of the patient P) but not aligned with spine of the patient P. For example, the third rotational axis 405 can be coplanar and perpendicular to the spine of the patient P. In this case, consider an imaging system 402 like that depicted FIG. 4 positioned at the foot of the table T (i.e., near the feet of the patient P).

The imaging system 402 can further include an image processing unit (not depicted) that processes the collected X-ray beam, collected as X-ray data, to form an image. Depending on the operation of the imaging system 402, the resulting image may be 2D (e.g., fluoroscopy), 3D (e.g., CBCT), or some other dimensionality (e.g., 4D, with consideration of temporal data). The imaging system 402 can further include a dedicated control system, that is, an imaging control system to control aspects of the imaging system 402 such as manipulation of the imaging system 402 through one or more of its degrees of freedom, the initiation and termination of an X-ray beam, and the flux of a generated X-ray beam. In some implementations, control of the imaging system 402 is performed by, or communicably coupled to, the control system 112 of the overarching medical system 100.

In some implementations, table T can provide one or more degrees of freedom. Degrees of freedom provided by table T may have the same relative effect with respect to the positioning and orientation of patient P relative to an imaging system 402, or elements of an imaging system (e.g., X-ray generation system 404 and X-ray collection system 406), as the degrees of freedom previously described with respect to imaging system 402. In some instances, degrees of freedom of the table T are used to position and orient patient P relative to the imaging system 402 in the absence of degrees of freedom provided by the imaging system 402 itself. For example, an imaging system 402 may be fixed with respect to one or more of the first and second translational degrees of freedom (408, 410). In these instances, table T may be translatable (e.g., using a rail system) to position the patient in a translational plane defined by the first and second translational DOFs (408, 410). In other instances, degrees of freedom provided by table T may be redundant in view of those provided by the imaging system 402 or may be used to extend the range of motion of one or more degrees of freedom provided by the imaging system 402. Table T can define a longitudinal axis (not depicted) that extends along the length of the table T, or from the “head-to-toe” of a patient P lying prone or supine on the table T. Similarly, table T can define a transverse axis (not depicted) that extends along the width of the table T, or from “shoulder-to-shoulder” of a patient P lying prone or supine on the table T. In some implementations, table T is rotatable about the transverse axis, where rotation of table T relative to the transverse axis raises or lowers the head or feet of patient P. That is, rotation of the table T relative to the transverse axis may be used to position a patient P that is lying prone or supine on the table T into the so-called Trendelenburg and reverse Trendelenburg positions. Likewise, in some implementations, table T is rotatable about the longitudinal axis, where rotation of table T relative to the longitudinal axis may tilt the right or left side of a patient P lying prone or supine on the table T up and down.

As depicted in FIG. 4, the medical system 100 may include a display system 110 for displaying an image or representation of the procedural site and the medical instrument 104. For example, the display system 110 can display an image representative of an anatomical structure and an inserted medical instrument 104, the image acquired using the imaging system 402. In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional (2D), two and a half-dimensional (2.5D), three-dimensional (3D), or higher-dimensional (e.g., including, for example, time based or velocity-based information) images. In some examples, one or more models are created from pre-operative or intra-operative image data sets and the virtual images are generated using the one or more models. In some instances, and as described below, intra-operative images can be used to locate and map (or track) the location of the medical instrument 104 as the medical instrument 104 moves through the patient anatomy.

The control system 112 of the medical system 100 may include a virtual visualization system that makes use of images acquired by the imaging system 402 to provide navigating and/or targeting assistance to operator O when controlling the medical instrument 104 during an image-guided medical procedure. In general, navigating and targeting sequences can be visualized using the visualization system based on an acquired pre-operative or intra-operative dataset of anatomic passageways of the patient P. The control system 112 or a separate computing device may convert images acquired with the imaging system 402, using programmed instructions alone or in combination with operator inputs, into a model of the patient anatomy. The model may include a segmented 2D, 2.5D, or 3D composite representation of a partial or an entire anatomic organ or anatomic region.

It is emphasized that while FIG. 4 depicts an example imaging system 402 as a mobile C-arm style imaging system capable of both CBCT (for 3D imaging) and fluoroscopy (for 2D imaging), embodiments of this disclosure are not limited to this type of imaging system 402 nor strictly require an imaging system 402. In general, embodiments disclosed herein can use any method for determining one or more physical parameters of a flexible elongate device (e.g., a shape sensor, position sensor, etc.) such that an imaging system 402 is not required in view of another method for determining the one or more physical parameters. In instances where an imaging system 402 is provided, the imaging system 402 can be used to determine one or more physical parameters of a flexible elongate device (either by itself or in conjunction with other methods) and/or validate aspects of the flexible elongate device (e.g., validate a restoration of the one or more physical parameters).

Embodiments disclosed herein generally relate to recording a value or state of one or more physical parameters of a flexible elongate device (e.g., flexible elongate device 202) and the restoration of the one or more physical parameters of the flexible elongate device to their recorded value or state in response to a determination that the flexible elongate device has exited the loss of actuation state. These physical parameters can include, but are not limited to: a position and/or orientation of the flexible elongate device or portion thereof (e.g., distal portion); a bend angle of a portion of the flexible elongate device; an orientation of an axis colinear or tangent to a portion (e.g., distal portion) of the flexible elongate device (e.g., a pointing direction); a distance from a target relative to the distal end of the flexible elongate device; a characterization of an apposition of a portion of the flexible elongate device or an apposition of an instrument inserted via the flexible elongate device (e.g., a contact force); and combinations thereof. For example, through operation of the medical system, the distal portion of the flexible elongate device can be pointed at a target (e.g., a peripheral pulmonary lesion), and a physical parameter of a pointing direction can be recorded to represent the pointing of the distal portion of the flexible elongate device at the target. As another example, through operation of the medical system, apposition can be established by a portion of the flexible elongate device and a physical parameter of a contact force (or an affirmative variable such as “yes” defined by the contact force exceeding a predefined threshold) can be recorded to represent the apposition state of the portion of the flexible elongate device.

During operation of the medical system, a flexible elongate device may enter a loss of actuation state where the shape of the flexible elongate device, or its pose (or one of its poses) or position and orientation of any portion thereof, including an apposition, is actively not maintained. During operation in a “following state” or a “hold state,” a manipulator assembly of a medical system may include a plurality of actuators (e.g., motors) that drive inputs on a medical instrument including the flexible elongate device in response to commands, such as from a control system of the medical system. These commands may be generated by user inputs during navigation or other operation of the flexible elongate device. The actuators may include drive systems that move the flexible elongate device in various ways when the medical instrument is coupled to the manipulator assembly. For example, one or more actuators may advance the flexible elongate device into a naturally or surgically created anatomic orifice. Further, actuators may control articulation of one or more articulable portions of the flexible elongate device, e.g., to move, steer, and/or hold the distal end (or any other portion) of the flexible elongate device in multiple degrees of freedom. Additionally, actuators can also be used to move an articulable end effector of the medical instrument including, or provided by, the flexible elongate device, such as for grasping tissue in the jaws of a biopsy device and/or the like. In some embodiments, the flexible elongate device may house cables (which may also be referred to as pull wires), linkages, or other actuation controls (not shown) that extend between its proximal and distal ends to controllably bend the distal end of the flexible elongate device. Thus, the flexible elongate device can be used to move or otherwise control tools (e.g., imaging tools, ablation tools, biopsy tools, electroporation tools, etc.) that are inserted within its flexible body. In general, actuators, cables, linkages, other actuation controls, or combinations thereof of the medical system (e.g., actuators of the manipulator assembly) are used to control the flexible elongate device and affect the configuration and/or shape of the flexible elongate device. The flexible elongate device can be controlled to realize a pose or shape (e.g., to establish apposition) and maintain or hold that pose or shape, e.g., through applied tension in cables using actuators. A hold state can refer to a control mode where the flexible elongate device is controlled or articulated to actively hold (or attempt to hold) a given shape or physical parameter such as a pose, a bend angle, pointing direction, apposition, etc. The hold state can include tightening pull wires to increase stiffness, as well as actively controlling pull wires to return to the hold position if there is deviation from external force. The following state indicates a control mode where a user is controlling the flexible elongate device for navigation or localization. This can include insertion, articulation, or rotation of the flexible elongate device.

The flexible elongate device possesses a stiffness property and, via active control (e.g., actuation, such as releasing of tension provided by pull wires) may revert to a “limp” state where no particular articulation or shape is specified by a user. The loss of actuation state, or the limp state, can also be described as a decrease in stiffness of the flexible elongate device, where the flexible elongate device is not considered stiff due to an applied actuation (e.g., to hold a pose such as in the hold state, to maintain apposition, etc.) but only possesses an inherent stiffness. In one or more embodiments, the loss of actuation state of the flexible elongate device is the same as this limp state. That is, in the loss of actuation state, the flexible elongate device may be not controlled to any specific pose, shape, or apposition, and, in general, may be allowed to articulate to an unspecified shape based on the physical stiffness properties of the flexible elongate device. Thus, in the loss of actuation state, a specified pose, shape, or apposition (or a controlled or actuated pose, shape, apposition, or, more simply, the pose or shape) of the flexible elongate device is not necessarily maintained. Once in the loss of actuation state, the flexible elongate device is more susceptible to changes in articulation and/or shape caused by external forces or physical properties of the device itself. However, the entry of the flexible elongate device into the loss of actuation state is caused by active control of the actuators (e.g., to be passive), which is different from changes of shape and/or pose that are caused only by external force (e.g., prolapse, deflections, etc.). That is, entering the loss of actuation state is not the same as observing a change in shape, apposition, and/or pose of the flexible elongate device, e.g., the change being a departure from a commanded shape, apposition, and/or pose, due to an external force.

The loss of actuation state, by definition of the shape of the flexible elongate device not being actively maintained, indicates that the one or more physical parameters associated with the flexible elongate device also may not be maintained. That is, in the loss of actuation state a particular shape (or a physical parameter) is not required. That said, while in the loss of actuation state, it may be possible that the shape (or a physical parameter) of the flexible elongate device does not change.

In accordance with one or more embodiments, a determination is made to enter the loss of actuation state for the flexible elongate device. As stated, in the loss of actuation state the shape and one or more physical parameters of the flexible elongate device are not actively maintained. Active maintenance can include both the following state and the holding state, as previously described, where actuators can be used to articulate the flexible elongate device, or portion thereof, or prevent articulation or movement of the flexible elongate device due to external forces. This could include tightening pull wires to increase stiffness, as well as actively controlling pull wires to return to the hold position if there is deviation from external force. In contrast, the determination to enter the loss of actuation state is considered an “active determination.” That is, although while in the loss of actuation state the flexible elongate device may be susceptible to changes in articulation and/or shape caused by external forces such that the shape of the flexible elongate device may change, these external forces are not said to cause the flexible elongate device to enter the loss of actuation state. Further, while an external force may cause an articulation or movement of the flexible elongate device, or portion thereof, such that the shape, apposition, and/or pose of the flexible elongate device changes (i.e., there is a change of a physical parameter), this behavior is not representative of the loss of actuation state, as described herein. That is, entry to the loss of actuation state is determined and not a consequence of an application of external forces. The determination to enter the loss of actuation state can be made, for example, in response to user input indicative of a command to enter the loss of actuation state. That is, in one or more implementations, a user (e.g., operator O) indicates or commands, e.g., using an input device provided at the master assembly, the flexible elongate device to enter the loss of actuation state. In other scenarios, the determination to enter the loss of actuation state is made in response to an observed fault or an occurrence of a specified event. Because a determination to enter the loss of actuation state is made, the loss of actuation state is distinguished from changes to the articulation and/or shape of the flexible elongate device that are caused only by external force.

Upon entering the loss of actuation state, the flexible elongate device may, for example, become limp and the distal portion of the flexible elongate device may move such that it is no longer pointing at the target or is no longer in apposition. Thus, in these examples, the pointing direction representative of the distal portion of the flexible elongate device pointing at the target or the apposition is not maintained. In accordance with one or more embodiments, and as explained in greater detail below, the one or more physical parameters is restored in response to a determination that the flexible elongate device has exited the loss of actuation state. Continuing with the current examples, in response to the determination that the flexible elongate device has exited the loss of actuation state the physical parameter of the pointing direction or apposition is restored to the flexible elongate device such that the distal portion of the flexible elongate device points at, or is directed at, the target, or is in apposition. In one or more embodiments, in response to the determination that the flexible elongate device has exited the loss of actuation state, the one or more physical parameters is restored to its value or respective state at some given time before the flexible elongate device entered the loss of actuation state (e.g., a recorded instance or a selected instance from a history of values or states).

A flexible elongate device can enter, or be determined to enter, the loss of actuation state for various reasons. Herein, the various reasons that a flexible elongate device enters, or is determined to enter, the loss of actuation state are categorized according to a cause, where the cause is one of: fault reaction logic of the medical system; an event-based inactive (“inactive”); and a user-initiated passive state (“passive”). A greater discussion of these causes is given below.

In one or more embodiments, the medical system 100 further includes a fault monitor (not shown), which may be provided by software, hardware, or a combination thereof to monitor for faults or failures in the medical system 100. In some embodiments, the fault monitor may be included as a sub-system or service of the control system 112 of FIG. 1. The fault monitor may provide a fault reaction logic supervisor that classifies faults. For example, faults may be classified as critical or noncritical. The fault monitor may operate to direct the medical system 100 and/or components thereof like the medical instrument system 200 to respond to the faults depending on their classification and/or other identifying characteristics of the faults. For example, the fault monitor may receive information from the sensor system 108 and identify discrepancies between information provided by redundant sensors. As another example, a fault can be triggered based on a quality indicator of the registration, such as an error value, that is used to determine whether the registration can be relied upon. In response to determination of a fault, and in some instances a classification thereof, fault reaction logic is applied to the medical system. The fault reaction logic can specify, according to the determined fault and/or classification thereof, an alteration to the operation and/or control of the medical system 100. For example, in response to a motion actuation fault the flexible body 216 of the flexible elongate device 202 can be locked in position such that forces acting upon it are actively resisted, i.e. actuators are activated to provide an opposing force in effort to maintain the pose of the flexible body 216. Various systems and methods for responding to faults in a robotic system, which may be applicable in some embodiments, are described in U.S. Patent Application Publication No. 2024/0065779 A1 (filed Oct. 20, 2023 and titled “Systems and methods for fault reaction mechanisms for medical robotic systems”), which is incorporated by reference herein in its entirety. In accordance with one or more embodiments, the flexible elongate device is caused to enter the loss of actuation state in response to a determined fault according to applied fault reaction logic. For example, the fault reaction logic can specify that the loss of actuation state is implemented in the flexible elongate device in response to a actuator error fault, where an actuator error fault indicates that an actuator used in the medical instrument system 200 is in error or otherwise not trustworthy. In other scenarios, the flexible elongate device is caused to enter the loss of actuation state as an inherit property of an occurrence of a fault in the medical system 100. For example, if the medical instrument system 200 experiences a loss of power, the flexible elongate device may not maintain its shape and enter the loss of actuation state. Thus, whether the loss of actuation state is implemented in response to a determined or detected fault as part of applied fault reaction logic or the loss of actuation state is an inherit property of a fault, it is said that the categorical cause of the loss of actuation state of the flexible elongate device is fault reaction logic.

An event-based inactive is described as an event that triggers a portion of the medical system to become inactive. Here, “inactivity” can include commanding the flexible elongate device to enter the loss of actuation state such that actuation (or activity) of the flexible elongate device is not permitted. That is, an event-based inactive can define an event that, if observed, causes the flexible elongate device to enter the loss of actuation state. An event, or the occurrence of an event, can be determined using one or more of a monitored parameter, condition, or state of the medical system. For example, an event can be determined by comparing a monitored parameter to a predefined threshold.

In contrast to the fault reaction logic, an event defined by an event-based inactive does not necessarily correspond to a fault detected in the medical system. An example of an event that can cause the flexible elongate device to enter the loss of actuation state is the state of one or more brakes disposed on a moveable cart that supports a manipulator assembly. As previously described, in one or more embodiments, the manipulator assembly of a medical system is provided on, or as, a movable cart. The moveable cart can be made stationary through activation of one or more brakes. A brake sensor can indicate a state (e.g., “on” or “off,” “active” or “inactive”) and/or quantitative measure of the one or more brakes (e.g., an applied breaking force, a normalized breaking force such as a range from 0% to 100%). Continuing with this example, in instances where the brake sensor indicates that the one or more brakes are in an “off” state or have transitioned from an “on” state to an “off,” a “cart brake off” event is observed and this event, based on an event-based inactive, can cause the flexible elongate device to enter the loss of actuation state. As another example, an event can relate to the state of a procedure performed by the medical system, where at various times during the procedure—indicated by the state of the procedure—it may be beneficial to deactivate a component of the medical system.

In one or more implementations, a flexible elongate device can enter the loss of actuation state in response to a user input, where the user input places the medical system into a user-initiated passive state (“passive”). For example, a medical system 100 can include a manipulator assembly 102 that supports and controls an instrument 104 including a flexible elongate device 202. The manipulator system and flexible elongate device 202 can be controlled by an operator O using a master assembly 106. As previously discussed, the master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, touchscreens (e.g., providing a graphical user interface), motion or presence sensors, and/or the like.

In one or more embodiments, the master assembly 106 is configured to receive a user input, using one or more included input devices, to activate the passive state of the medical system 100 including entering the loss of actuation state for the flexible elongate device 202. For example, in one or more implementations, the master assembly 106 includes a button for activating and deactivating the passive state and thus for entering and exiting the loss of actuation state of the flexible elongate device. The button can be pressed to cause the flexible elongate device to enter the loss of actuation state and then pressed again to cause the flexible elongate device to exit the loss of actuation state. In one or more embodiments, the master assembly 106 includes a button (e.g., a passive button) configured to receive a user input that controls the loss of actuation state of the flexible elongate device. In some implementations, the button can execute various command depending on a type of input received with the button. The types of input can include, but are not limited to, a distinct press and release of the button, a press and hold of the button, and multiple sequential presses and releases of the button in a given period (e.g., a double click). That is, the button can exhibit a function overloading behavior where the same button is used to execute or initiate multiple functions. In one or more embodiments, the button is configured such that a single press (i.e., a quick and distinctive press and release) toggles the loss of actuation state (e.g., entering the loss of actuation state if not in the loss of actuation state and exiting from the loss of actuation state if in the loss of actuation state) and a press and hold of the button either slowing enters the loss of actuation state or exits the loss of actuation state and slowly restores the one or more physical parameters to the flexible elongate device depending on the state of the flexible elongate device. For example, in a scenario where the flexible elongate device is not in the loss of actuation state, the button can be pressed and held to command the flexible elongate device to slowly (e.g., over the course of 5 seconds) enter the loss of actuation state where physical parameters of the flexible elongate device (e.g., pointing direction, apposition) are actively not maintained. That is, in entering the loss of actuation state the flexible elongate device becomes limp with reduced stiffness. Thus, slowing entering the loss of actuation state can include reducing, over a defined period of time (e.g., 5 seconds), the stiffness of the flexible elongate device (e.g., removing tension in cables by backdriving actuators). Continuing with this example, the button can be pressed and held resulting in a determination to exit the loss of actuation state of the flexible elongate device and to restore one or more physical parameters to the flexible elongate device. Restoration of one or more physical parameters to the flexible elongate device in response to a determination to exit the loss of actuation state is described in greater detail later in the instant disclosure.

The medical system 100 can also include a display system 110. In one or more embodiments, the above-described behavior of a passive button of the master assembly 106 is implemented with, or in conjunction with, the display system 110. For example, the display system 110 can include a touch screen configured to receive one or more user inputs. In this example, the passive button can be provided using the touch screen of the display system 110 as opposed to, or in addition to, an input device of the master assembly 106. As another example, the master assembly 106 can include an input device that is used to navigate a menu and cause the flexible elongate device to enter or exit the loss of actuation state via a user selection, where the menu and selectable item(s) are displayed to the operator O using the display system 110. As described later in the instant disclosure, the display system 110 can also be used to display alerts and/or information to the operator O regarding the state of the medical system 100 and flexible elongate device 202, for example, relating to received user inputs using one or more input devices of the master assembly 106 or display system 110 itself.

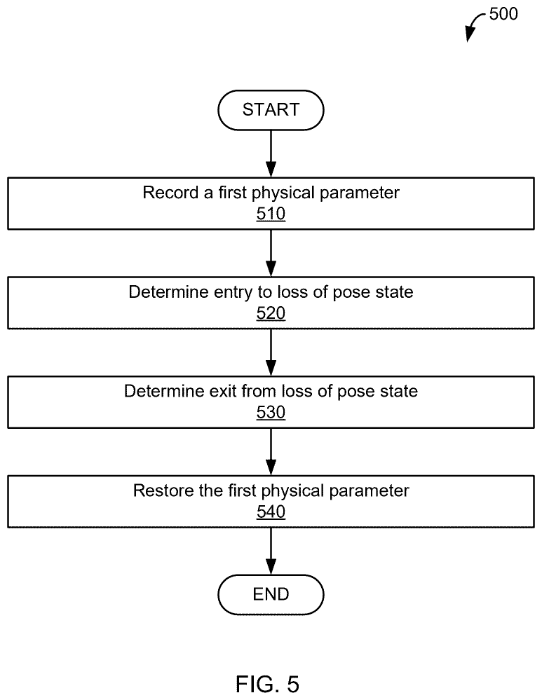

FIG. 5 depicts a method 500 for entering a loss of actuation state for a flexible elongate device 202 as part of, or controlled by, a medical system 100 (e.g., using a manipulator assembly 102), exiting the loss of actuation state, and restoring one or more physical parameters to the flexible elongate device 202 that were actively not maintained in the loss of actuation state. The method may be implemented using instructions stored on a non-transitory medium that may be executed by a computing system, e.g., the control system 112.

In Block 510, a first physical parameter of the flexible elongate device 202 is recorded and stored, e.g., using the control system 112. Examples of the first physical parameter can include: a position and/or orientation of the flexible elongate device 202 or portion thereof; a bend angle of a portion of the flexible elongate device; an orientation of an axis colinear or tangent to a portion of the flexible elongate device such as the distal portion (e.g., a pointing direction); a distance from a target relative to the distal end of the flexible elongate device; any parameter associated with apposition (e.g., as previously discussed), and others not listed. The first physical parameter is recorded while the flexible elongate device 202 is not in the loss of actuation state. Embodiments disclosed herein can use one or more of a shape sensor, a position sensor, a contact force sensor, and intra-operative image data (e.g., macro-level, or externally acquired, images of the anatomy and/or internally acquired images) to determine and record the first physical parameter of the flexible elongate device 202.

In some implementations, the first physical parameter is recorded automatically by the medical system 100 (e.g., control system 112), e.g., according to a predefined frequency or period. For example, the first physical parameter can be recorded every 1 second. In other implementations, the first physical parameter is recorded—while the flexible elongate device 202 is not in the loss of actuation state—in response to a determination that the first physical parameter has changed since its last recorded state, where the change is determined using a change function and may further be compared to a threshold. In these implementations, one or more of the change function and threshold can depend on the type of the first physical parameter (i.e., a different threshold for a pointing direction than for a position of the distal end of the flexible elongate device) and/or other factors of the medical system 100 such as the state of a procedure. For example, in instances where the first physical parameter is a position of the distal end or tip of the flexible elongate device 202 given using Cartesian coordinates x, y, and z, the change function may be

$${Δ}_{p o s} = {( {( {x}_{i} - {x}_{i - 1} )}^{2} + {( {y}_{i} - {y}_{i - 1} )}^{2} + {( {z}_{i} - {z}_{i - 1} )}^{2} )}^{\frac{1}{2}} , \qquad EQ 1$$

where Δₚₒₛ is the determined change in the position of the distal end of the flexible elongate device 202, the subscript i indicates the current value of its associated coordinate, and the subscript i−1 indicates the last recorded value of its associated coordinate. In this example, in instances where

$${Δ}_{p o s} > {Δ}_{p o s}^{theshold} ,$$

the current position of the distal end of the flexible elongate device can be recorded and subsequently referenced in EQ 1 using the subscript i−1.

As stated above, one or more of the change function and threshold, if used, can be dependent on a state of the medical system 100 and/or a state of a procedure being executed using the medical system 100. For example, in the case of a pointing direction, in some implementations, an associated threshold can depend on whether a navigation procedure or a localization procedure is being performed. In some implementations, the pointing direction threshold is set to tolerate larger changes between the current and last recorded pointing directions when performing a navigating procedure than when performing a localization procedure.

In other implementations, the first physical parameter (i.e., Block 510) is recorded in response to a user input. For example, a user input can include a command to enter the loss of actuation state for the flexible elongate device. The user input can be received using a passive button provided on one or more of the master assembly 106, display system 110, or other component of the medical system 100, as previously described. That is, the first physical parameter can be recorded in response to a user-initiated passive state or other user input.

In one or more implementations, various values or states for the first physical parameter are recorded and stored, e.g., with respect to time, forming a recorded history.

Keeping with the method 500 of FIG. 5, in Block 520 an entry to the loss of actuation state for the flexible elongate device 202 is determined. This determination can be made in view of, or caused by, fault reaction logic, an event-based inactive, or a user-initiated passive state. In one or more implementations, the execution of Block 510 to record the first physical parameter occurs in response to the determination to enter the loss of actuation state of Block 520. For example, the medical system 100 can receive a user input (e.g., pressing a passive button on the master assembly 106) to enter the loss of actuation state for the flexible elongate device 202. In response to the received user input, the first physical parameter can be recorded and then the flexible elongate device 202 can be placed in the loss of actuation state. In other implementations, the determination to the enter the loss of actuation state can immediately place the flexible elongate device 202 in the loss of actuation state. In these implementations, the first physical parameter is said to have been previously recorded, e.g., according to a given period or change function.

Entry into the loss of actuation state includes actively placing the flexible elongate device 202 into a limp state where no particular articulation or shape (including apposition) is specified by a user (e.g., operator O). Entry into the loss of actuation state may include placing one or more actuators used to control the flexible elongate device 202 in a passive mode where the one or more actuators are freely driven in response to an externally applied force (e.g., a body force such as gravity). In other scenarios, entry to the loss of actuation state includes actively backdriving one or more actuators used in the control of the flexible elongate device 202 to remove tension from, or induce slack in, the flexible elongate device 202. Entry of the flexible elongate device into the loss of actuation state is caused by active control of the actuators, which is different from changes of shape and/or pose that are caused only by external force (e.g., prolapse, deflections, etc.).

In Block 530, a determination to exit from the loss of actuation state for the flexible elongate device 202 is made. The determination to exit from the loss of actuation state may depend on the determination to enter the loss of actuation state or a specific cause. For example, in instances where the determination to enter the loss of actuation state was caused by, or as a part of, fault reaction logic, then the determination to exit the loss of actuation state may require that that the initiating fault be resolved. In some implementations, the resolution of all faults that are determined to place the flexible elongate device 202 into the loss of actuation state automatically triggers the determination to the exit the loss of actuation state. In other implementations, an option to exit the loss of actuation state for the flexible elongate device is provided to an operator (e.g., using the display system 110) in response to a resolution of one or more faults. As another example, in instances where the determination to enter the loss of actuation state was caused by an event-based inactive (e.g., cart brake detected as “off”), the determination to exit the loss of actuation state can be made in response to another event based on the inactive (e.g., cart brake restored to “on”). Similar to the cases when the determination to enter the loss of actuation state is caused, directly or indirectly, by fault reaction logic, the determination to exit the loss of actuation state having entered the loss of actuation state based on an inactive can be made automatically or with user input, e.g., using a prompt to the operator. As yet another example, the determination to exit the loss of actuation state can be made, or caused by, the deactivation of the user-initiated passive state. That is, in instances where the flexible elongate device 202 is entered into the loss of actuation state through user input (e.g., using a passive button), user input can also be used to exit the loss of actuation state. The determination to exit the loss of actuation state restores control functionality (e.g., steering) to the flexible elongate device 202. However, upon exiting the loss of actuation state, the flexible elongate device 202 can have a different pose, shape, or apposition than before entering the loss of actuation state. In some implementations, exiting from the loss of actuation state returns, or places, the flexible elongate device 202 in a hold state or a following state 202.

In Block 540, the first physical parameter is restored to the flexible elongate device 202. Restoration of the first physical parameter includes controlling, automatically, the flexible elongate device 202 to move, as necessary, so that the first physical parameter has the same value or state as was recorded prior to entering the loss of actuation state. As previously stated, in some implementations a history of recorded first physical parameters exists. In these implementations, a user can select the value or state for the first physical parameter from the history or, alternatively, the value or state recorded immediately prior to the determination to enter the loss of actuation state can be used.

Notably, restoration of the first physical parameter is not generally realized by restoring the state or positions of the one or more actuators that are used in the control of the flexible elongate device 202. In general, having entered and exited the loss of actuation state, a hysteresis behavior is exhibited in the flexible elongate device 202 with respect to the first physical parameter. As such, the first physical parameter is not guaranteed to be restored by, for example, reversing processes or actuator states observed while entering the loss of actuation state.

As discussed above, embodiments disclosed herein can use one or more of a shape sensor, a position sensor, a contact force sensor, and intra-operative image data (e.g., macro-level, or externally acquired, images of the anatomy and/or internally acquired images) to determine and store one more physical parameters of the flexible elongate device 202. Similarly, one or more of a shape sensor, a position sensor, a contact force sensor, and intra-operative image data can be used to restore the first physical parameter to its previously recorded value or state. For example, in instances where a shape sensor is used to determine the shape of the flexible elongate device 202 and thus the first physical parameter, restoration of the first physical parameter can include controlling the flexible elongate device 202 to restore the recorded value or state for the first physical parameter as guided by the shape sensor. That is, restoration of the first physical parameter can include a determination of the current shape of the flexible elongate device, e.g., having exited from the loss of actuation state, using the shape sensor and determining a trajectory or movement for the flexible elongate device 202 to restore the first physical parameter. Then, the flexible elongate device 202 can be controlled according to the trajectory or movement. Further, the trajectory or movement can be monitored and/or validated using the shape sensor.

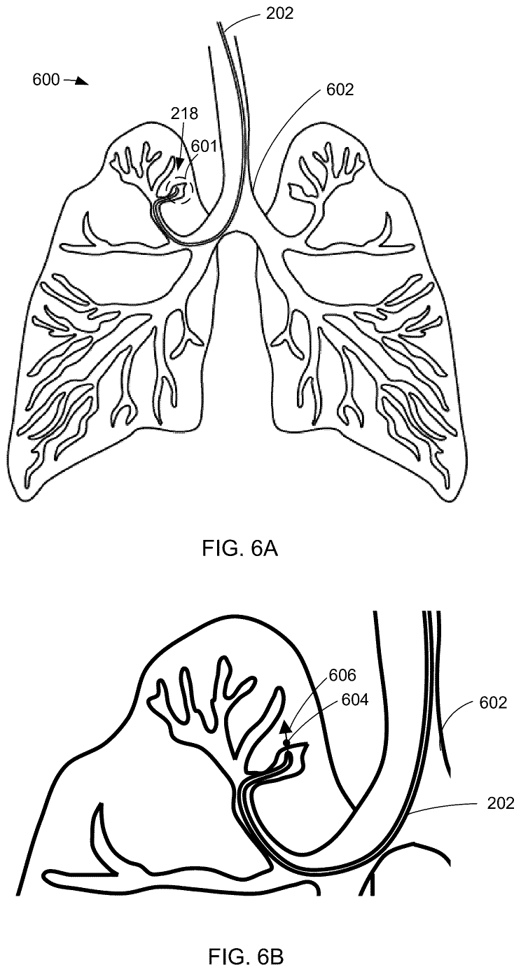

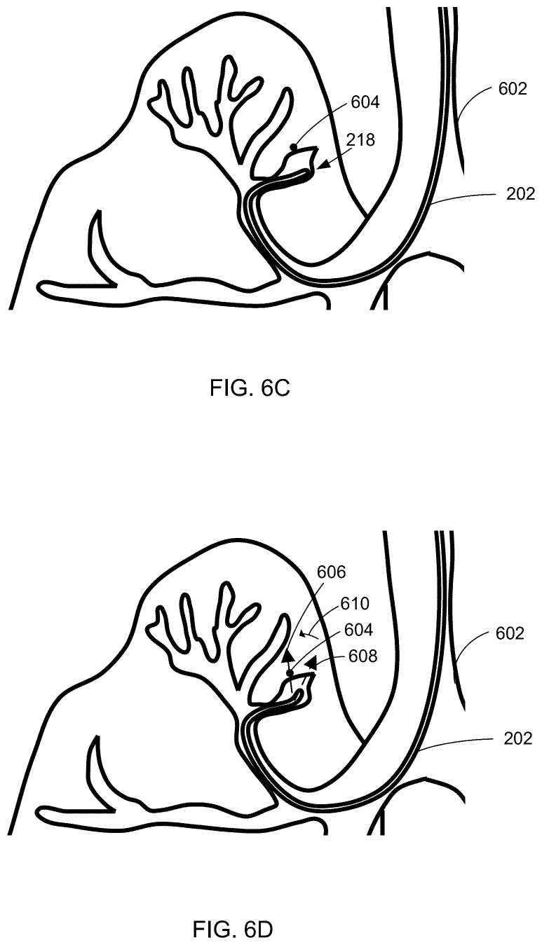

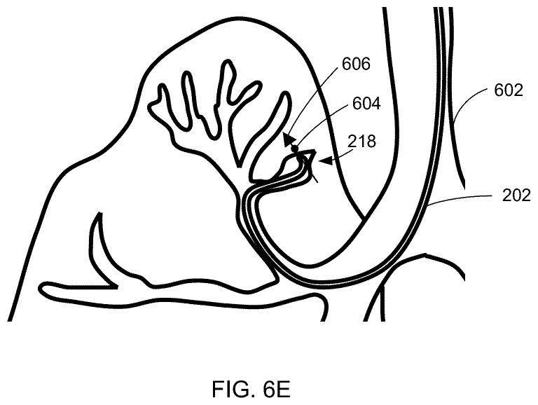

FIGS. 6A-6D depict an example application of the method of FIG. 5, in accordance with one or more embodiments. FIG. 6A depicts a flexible elongate device 202 having been advanced through anatomic passageways 602 of lungs 600. These passageways 602 include the trachea and the bronchial tubes. In the depicted example, the flexible elongate device 202 has been navigated such that its distal end 218 is at a target region 601. FIGS. 6B-6D depict enlarged views of the distal end 218 of the flexible elongate device 202 in the anatomic passageways 602 of the lungs 600 of FIG. 6A. In particular, FIGS. 6B-6D depict a target 604 or target structure which may be, for example, a peripheral pulmonary lesion (PPL). In navigating through the anatomic passageways 602, the flexible elongate device 202 assumes a shape. The shape can be measured using one or more of a shape sensor, a position sensor, and intra-operative imaging techniques. In FIG. 6B, the shape of the flexible elongate device 202 is used to determine one or more physical parameters of the flexible elongate device 202. In the given example, a first physical parameter of a pointing direction 606 is determined using shape information of the flexible elongate device 202 registered to images and/or a model of the lungs 600. The pointing direction 606 can include an orientation of an axis that is colinear with a distal portion of the flexible elongate device 202. In the example of FIG. 6B, the pointing direction 606 is such that the distal end of the flexible elongate device 202 is pointed, or directed, at the target 604. In other words, FIG. 6B depicts a scenario where the first physical parameter of a pointing direction 606 has been determined and recorded, and the recorded pointing direction 606 has the distal end of the flexible elongate device 202 pointing at the target 604.

In FIG. 6C, the flexible elongate device 202 is said to have entered the loss of actuation state. The flexible elongate device 202 can be caused to enter the loss of actuation state by one or more of fault reaction logic, an event-based inactive, and a user-initiated passive state. In the loss of actuation state, the flexible elongate device 202 becomes limp and the shape of the flexible elongate device 202 is actively not maintained. Further, in the loss of actuation state, the first physical parameter is actively not maintained. That is, in the loss of actuation state, the first physical parameter can change value or state compared to a recorded value or state. Entry of the flexible elongate device 202 into the loss of actuation state is caused by active control of the actuators, which is different from changes of shape and/or pose that are caused only by external force (e.g., prolapse, deflections, etc.). In FIG. 6C, while in the loss of actuation state, the distal end of the flexible elongate device 202 is seen to move such that its distal end is no longer directed toward the target 604.

In FIG. 6D, it is said that the flexible elongate device 202 has exited the loss of actuation state. In accordance with one or more embodiments, the flexible elongate device 202 is controlled to restore the first physical parameter (or pointing direction 606), as previously recorded (FIG. 6B). FIG. 6D depicts a current pointing direction 608 of the flexible elongate device 202 and the recorded pointing direction 606. In one or more embodiments, restoration of the first physical parameter includes determining a trajectory, or planned movement, for the distal end of the flexible elongate device 202 such that the current pointing direction 608 aligns with, or is substantially equal to, the recorded pointing direction 606. FIG. 6D depicts movement 610 of the distal end of the flexible elongate device 202, e.g., according to a determined trajectory, to restore the first physical parameter of, in this case, a pointing direction 606. Notably, restoration of the one or more physical parameters for the flexible elongate device (or portion thereof such as an articulable body portion) does not require that the shape of flexible elongate device, or portion thereof, be the same as before the flexible elongate device was determined to enter the loss of actuation state. For example, in one or more implementations, the pointing direction can generally be defined using an axis that is colinear to the distal portion of the flexible elongate device. However, the stored pointing direction can further store or make use of a position of a point or object, e.g., a target, and a distance of the distal end of the flexible elongate device to that position. For example, the stored pointing direction can indicate that the axis that is colinear to the distal portion of the flexible elongate device intersects a target and that the distal end of the flexible elongate device is at, or within, a distance from the target. Then, in restoring the pointing direction to the stored pointing direction, the distal portion of the flexible elongate device can have a different position and orientation relative to the target but the physical parameter of pointing direction can still be considered restored if the axis that is colinear to the distal portion of the flexible elongate device intersects the target and the distal end of the flexible elongate device is within a distance threshold from the target. That is, if a stored pointing direction has the distal end of the flexible elongate device directed at the target, a restored pointing direction can include various positions and orientations for the distal end of the flexible elongate device as long as the distal end is directed at the target. In this way, the stored pointing direction may better be viewed as a state, where a state can specify that the distal end of the flexible elongate device 202 is in a state of pointing at a specified target, rather than a value (e.g., a position vector). FIG. 6E depicts an example where the pointing direction 606 is restored without the flexible elongate device 202, or portion thereof, assuming its position and/or orientation before entering the loss of actuation state. That is, while FIG. 6E depicts a restored pointing direction 606, the position and/or orientation of the distal end of the flexible elongate device is not the same as depicted in FIG. 6B. In FIG. 6E, the pointing direction 606 is said to be restored because the distal end of the flexible elongate device 202 is directed, or pointed, at the target 604.

Restoration of one or more physical parameters can be monitored and validated, e.g., using shape information of the flexible elongate device 202. As discussed, the shape of the flexible elongate device 202, or portion thereof, can be determined using one or more of a shape sensor, a position sensor, and intra-operative imaging techniques. In one or more embodiments, a trajectory or movement plan for restoring one or more physical parameters of the flexible elongate device 202 is determined using the current shape of the flexible elongate device 202 (i.e., current values or states for one or more physical parameters) and the recorded values or states for the one or more physical parameters to be restored. As an example, a physical parameter to be restored can be a position of the distal end of the flexible elongate device 202. Given the current position of the distal end of the flexible elongate device 202, as determined by or as a part of the current shape of the flexible elongate device 202, a trajectory can be determined that minimizes the difference between the current position and the desired position of the distal end of the flexible elongate device.

Restoration of the one or more physical parameters can be subject to various constraints. For example, the flexible elongate device 202 can be controlled using one or more actuators. The one or more actuators can each include a torque or force sensor or an approximator for determining torque or force. The amount of torque or force applied by an actuator can indicate an amount of resistance experienced by the flexible elongate device 202. Thus, a relatively high force or torque observed by an actuator can correspond to a force applied to a portion of the flexible elongate device 202. The force applied to a portion of the flexible elongate device 202 can originate from a contact between the flexible elongate device 202 and its environment, e.g., patient anatomy. As such, it may be desirable to limit the torque or force than can be applied to by actuator used for control or articulation of the flexible elongate device 202. In accordance with one or more embodiments, during restoration of the one or more physical parameters, the force or torque of one or more actuators used to control the flexible elongate device 202 is monitored and constrained to predefined threshold values. In one or more implementations, movements of the flexible elongate device 202 that result in actuator torque or force values that exceed predefined thresholds are prohibited during restoration of the one or more physical parameters for the flexible elongate device 202. Other constraints, such as a maximum allowable departure of a portion of the flexible elongate device 202 from a prescribed trajectory, can be applied without departing from the scope of the instant disclosure.

The process of restoring the value or state of the one or more physical parameters of the flexible elongate device 202 to a recorded value or state, where this process includes movement of the flexible elongate device 202, can be interrupted by one or more interrupts. The interrupts can include, but are not limited to, violation of a constraint as described above, observation of an event-based inactive or other event, change in a state of the medical system or procedure performed using the medical system, reception of a user input indicative of a command to pause or abort the restoration process, and reception of a user input to assume control of the flexible elongate device 202. For example, in some implementations, if the one or more physical parameters cannot be restored, automatically, without violating a constraint the restoration process is aborted. Abortion of the restoration process can include pausing motion or automated control of the flexible elongate device 202, placing the flexible elongate device 202 into the loss of actuation state, raising an alert to a user, or combinations thereof. As another example, an observed event of a cart brake switching from “on” to “off” can cause the restoration process to abort. As yet another example, an operator can interact with the master assembly 106 to control the flexible elongate device 202. In this scenario, the user input to control the flexible elongate device 202 is received and processed by the control system 112 and manipulator assembly 102 to control the flexible elongate device 202 and the restoration of the one or more physical parameters is aborted. As yet another example, a change in a state of a medical tool 226 conveyed by the flexible elongate device 202 can cause a pause or abortion of the restoration process. For example, the medical tool 226 can be a biopsy needle. The distal end of the biopsy needle can be enclosed by the flexible elongate device 202 or moved to protrude from the distal end of the flexible elongate device 202, e.g., to contact a target. In one or more implementations, the state of the biopsy needle as either “enclosed” in or “protruding” from the flexible elongate device 202 is determined and/or monitored. In instances where the biopsy needle is protruding from the flexible elongate device 202, the restoration of the one or more physical parameters for the flexible elongate device 202 can be prevented or aborted.

In accordance with one or more embodiments, the medical system 100 is configured to provide various alerts to the operator O and/or other users of the medical system 100. The alerts can include any combination audio signals, visual signals (e.g., flashing light), feedback forces, vibrations or tactile forces, display of messages (e.g., using the display system 110), etc. An alert such as a displayed message or icon can be provided to the operator O indicative of whether the flexible elongate device 202 is in the loss of actuation state. Further, in the case of an interrupt during the restoration process, the alert can indicate the nature, type, or cause of the interrupt and provide the operator O with one or more options for overcoming the interrupt (e.g., dismissal of the interrupt, implementation of a corrective action, etc.). For example, during the restoration process a constraint such as an actuator torque limit can be violated. In this example, the restoration process is paused (i.e., automatic movement of the flexible elongate device 202 is paused), a brief audio tone or beep is emitted to indicate that the restoration process has been paused, and a message is displayed to the operator O that the one or more physical parameters cannot be automatically restored and that the operator O must assume control of the flexible elongate device 202 to navigate or localize the flexible elongate device 202 as desired.

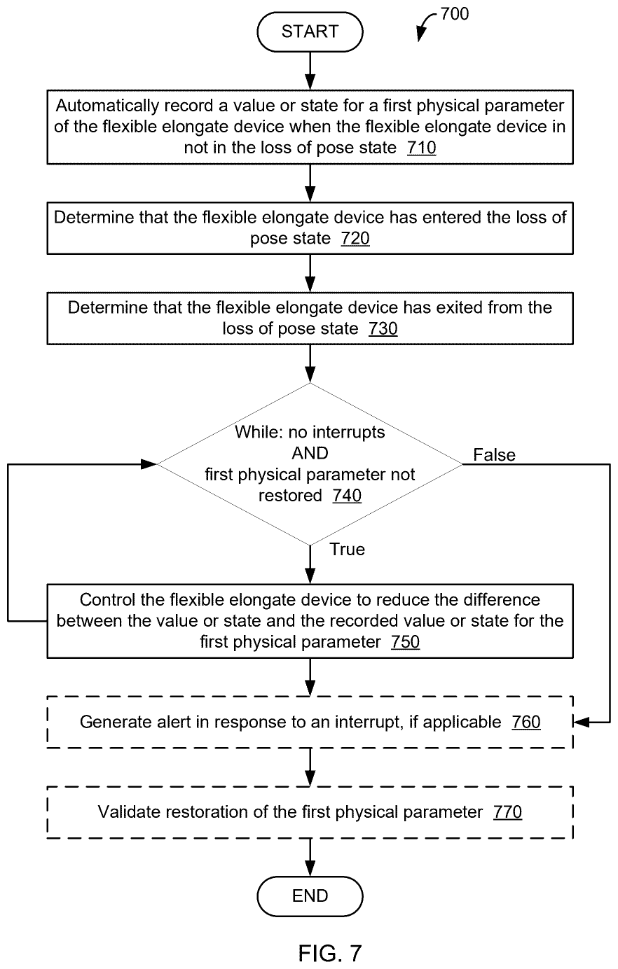

FIG. 7 depicts a flowchart 700 for restoring one or more physical parameters for a flexible elongate device 202, the flexible elongate device 202 having entered and exited the loss of actuation state. The flexible elongate device 202 can be controlled by a control system 112 of a medical system 100 (e.g., using a manipulator assembly 102). In Block 710, a value or state for at least a first physical parameter of the flexible elongate device is automatically recorded. Notably, the value or state of the first physical parameter is recorded while the flexible elongate device is not in the loss of actuation state. While FIG. 7 references a first physical parameter of the flexible elongate device 202, additional physical parameters such as a second physical parameter and a third physical parameter can be recorded without limitation. That is, the flowchart 700 of FIG. 7 is applicable to any number of physical parameters for the flexible elongate device 202. Examples of the first physical parameter can include: a position and/or orientation of the flexible elongate device 202 or portion thereof; a bend angle of a portion of the flexible elongate device 202; an orientation of an axis colinear or tangent to a portion of the flexible elongate device 202 such as the distal portion (e.g., a pointing direction); a distance from a target relative to the distal end of the flexible elongate device 202; and others not listed, such as previously discussed apposition-related parameters. As previously discussed, the first physical parameter—or, more specifically, a value or state for the first physical parameter—can be recorded, automatically, based on a schedule (e.g., a period or frequency) or in response to a trigger such as the result of a change function exceeding a given threshold. The first physical parameter, or value or state of the first physical parameter, can be determined using one or more of a shape sensor, a position sensor, and intra-operative imaging.

In Block 720, it is determined, for example, using the control system 112, that the flexible elongate device 202 has entered the loss of actuation state. As previously described, the loss of actuation state is characterized by a decreased stiffness of the flexible elongate device 202. In particular, in the loss of actuation state, the shape of the flexible elongate device 202 is actively not maintained and no particular articulation or shape is specified by a user. While in the loss of actuation state, the flexible elongate device 202, or portions thereof, can move, e.g., as a result of gravity, however, the application of external forces to move the flexible elongate device 202 is not said to place the flexible elongate device in the loss of actuation state. Entry to the loss of actuation state is made through active determination. That is, entry of the flexible elongate device 202 into the loss of actuation state is caused by active control of the actuators, which is different from changes of shape and/or pose that are caused only by external force (e.g., prolapse, deflections, etc.). In association with the shape of the flexible elongate device 202 not being maintained, the value or state of the first physical parameter is also actively not maintained. Thus, in the loss of actuation state the flexible elongate device 202 may move resulting in a departure of the first physical parameter from its recorded value or state.

In Block 730, it is determined, for example, using the control system 112, that the flexible elongate device 202 has exited the loss of actuation state. Exit from the loss of actuation state includes providing control or articulation of the flexible elongate device 202 using the manipulator assembly 102. Exit from the loss of actuation state can include returning, or placing, the flexible elongate device in a hold or following state. Having exited the loss of actuation state, the value or state of the first physical parameter may not be the same as the recorded value or state (see Block 710).

Block 740 of the flowchart 700 represents a decision evaluated according to two conditions. A first condition checks whether the control system 112 has raised or received (e.g., from another component of the medical system 100 such as the manipulator assembly 102) an interrupt. Interrupts can be generated for a variety of reasons, including, but not limited to: a violation of a constraint (e.g., torque limit); an observation of an event-based inactive or other event (e.g., cart brake being “off”); a change in a state of the medical system or procedure performed using the medical system (e.g., biopsy needle in “extended” or “protruding” position); a reception of a user input indicative of a command to pause or abort a restoration process; and reception of a user input to assume control, by an operator O, of the flexible elongate device 202. The second condition checks whether the value or state of the first physical parameter has been restored to the recorded value or state for the first physical parameter. The first physical parameter is considered restored when its value or state is equal to (or within a given tolerance) the recorded value or state. If both the first and second conditions of the decision Block 740 are true, then the flowchart 700 of FIG. 7 proceeds to Block 750. In Block 750, the flexible elongate device 202 is controlled, for example, using the control system 112 and manipulator assembly 102, to reduce a difference between the value or state and recorded value or state of the first physical parameter. That is, Block 750 represents the restoration process where one or more physical parameters (including the first physical parameter) is restored to a previously recorded value or state. Control of the flexible elongate device 202 to restore the first physical parameter is performed without user input. That is, the flexible elongate device 202 is made to move automatically. Restoration of the first physical parameter can include determining a trajectory or movement plan for the flexible elongate device 202, or portion thereof (e.g., articulable portion). If at least one of the first and second conditions of decision Block 740 is false, the flowchart 700 of FIG. 7 proceeds to Block 760.

Block 760 is considered optional and its optionality is indicated with a dashed border. That is, in one or more implementations, Block 760 is omitted from the flowchart 700. Block 760 corresponds to the first condition of decision Block 740 and is only applied if an interrupt was generated during the restoration process (i.e., during execution of Block 750). If an interrupt was observed or raised during the restoration process, Block 760 indicates that an alert is generated in response to the interrupt. The alert can include any combination of audio, visual, tactile, and force signals to a user. For example, the alert an include a message displayed to the user using the display system 110 where the message provides information regarding the interrupt such as a type of the interrupt and potential corrective actions. A user response to Block 760 can result in the termination of the flowchart 700 or can return execution of the flowchart 700 to any previous block.

Similar to Block 760, Block 770 is also considered optional and is likewise indicated as such with a dashed border. Thus, in one or more implementations, Block 770 is omitted from the flowchart 700. In Block 770, restoration of the first physical parameter is validated. Validation can include determining a value or state for the first physical parameter, e.g., using a shaper sensor of the flexible elongate device 202, and ensuring that the value or state for the first physical parameter is within a prescribed tolerance of the recorded value or state. If validation of the restoration of the first physical parameter fails, the flowchart 700 can return to a previous block or be terminated. For example, in some implementations, failure to validate the restoration of the first physical parameter causes an alert to be generated and the flowchart 700 returns to Block 770. In one or more implementations, validation is performed concurrently with the restoration process of Block 750. That is, Block 750 is executed to restore the first physical parameter and the decision Block 740 terminates the while loop when the first physical parameter is determined to be restored, i.e., the second condition is false.

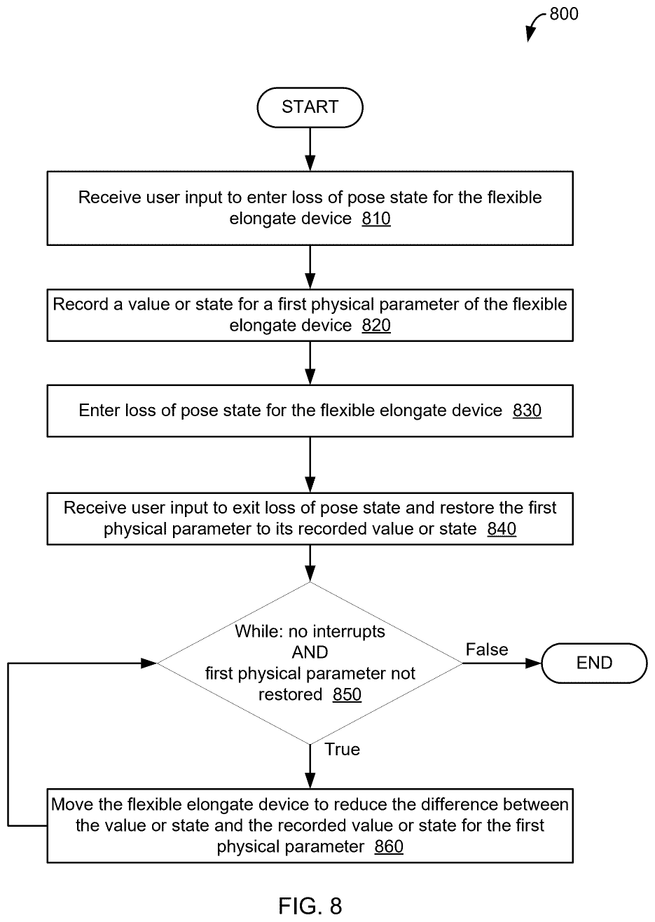

FIG. 8 depicts a flowchart 800 for restoring one or more physical parameters for a flexible elongate device 202, where the flexible elongate device 202 is controlled to enter and exit the loss of actuation state by user input. The flexible elongate device 202 can be controlled by a control system 112 of a medical system 100 (e.g., using a manipulator assembly 102). In Block 810, user input is received to enter the loss of actuation state for the flexible elongate device 202. For example, the user input can be received using an input device at the master assembly 106 and/or display system 110 of the medical system 100. In one or more implementations, the master assembly 106 includes a button, described here as a passive button, for receiving inputs related to the loss of actuation state for the flexible elongate device 202.

In Block 820, in response to receiving the user input to enter the loss of actuation state, a value or state for at least a first physical parameter of the flexible elongate device 202 is recorded. Notably, the value or state of the first physical parameter is recorded before the flexible elongate device 202 is caused to enter the loss of actuation state.

In Block 830, the flexible elongate device 202 is caused to enter the loss of actuation state. As previously described, the loss of actuation state is characterized by a decreased stiffness of the flexible elongate device 202. In particular, in the loss of actuation state, the shape of the flexible elongate device 202 is actively not maintained and the flexible elongate device 202, or portions thereof, can move, e.g., as a result of gravity. Entry of the flexible elongate device into the loss of actuation state is caused by active control of the actuators, which is different from changes of shape and/or pose that are caused only by external force (e.g., prolapse, deflections, etc.). In association with the shape of the flexible elongate device 202 not being maintained, the value or state of the first physical parameter is also actively not maintained. Thus, in the loss of actuation state the flexible elongate device 202 may move resulting in a departure of the first physical parameter from its recorded value or state.

In Block 840, user input is received to exit the loss of actuation state for the flexible elongate device 202. For example, the user input can be received using an input device at the master assembly 106 and/or display system 110 of the medical system 100. Exit from the loss of actuation state includes providing control or articulation of the flexible elongate device 202 using the manipulator assembly 102. Exit from the loss of actuation state can include returning, or placing, the flexible elongate device 202 in a hold state or a following state. Having exited the loss of actuation state, the value or state of the first physical parameter may not be the same as the recorded value or state (see Block 820).

Block 850 of the flowchart 800 represents a decision evaluated according to two conditions. A first condition checks whether the control system 112 has raised or received (e.g., from another component of the medical system 100 such as the manipulator assembly 102) an interrupt. Interrupts can be generated for a variety of reasons, including, but not limited to: a violation of a constraint (e.g., torque limit); an observation of an event-based inactive or other event (e.g., cart brake being “off”); a change in a state of the medical system or procedure performed using the medical system (e.g., biopsy needle in “protruding” position); a reception of a user input indicative of a command to pause or abort a restoration process; and reception of a user input to assume control, by an operator O, of the flexible elongate device 202. The second condition checks whether the value or state of the first physical parameter has been restored to the recorded value or state for the first physical parameter. The first physical parameter is considered restored when its value or state is equal to (or within a given tolerance) the recorded value or state.

If at least one of the first and second conditions of the decision Block 850 are false, then, in one or more implementations, the flowchart 800 of FIG. 8 terminates. In other implementations, the flowchart 800 of FIG. 8 can include additional blocks (not shown) such as Block 760 and Block 770 of FIG. 7 relating to generating alerts and validating the restoration of the first physical parameter, respectively. Such additional blocks, if included, can be executed in response to the decision Block 850 evaluating to false.

If both the first and second conditions of the decision Block 850 are true then the flowchart 800 of FIG. 8 proceeds to Block 860. In Block 860, the flexible elongate device 202 is controlled, for example, using the control system 112 and manipulator assembly 102, to reduce a difference between the value or state and recorded value or state of the first physical parameter. That is, Block 860 represents the restoration process where one or more physical parameters (including the first physical parameter) is restored to the previously recorded value or state. Control of the flexible elongate device 202 to restore the first physical parameter is performed without user input. That is, the flexible elongate device 202 is made to move automatically. Restoration of the first physical parameter can include determining a trajectory or movement plan for the flexible elongate device 202, or portion thereof (e.g., articulable portion).

In one or more implementations, the master assembly 106 includes a passive button used to receive the user inputs for Block 810 and Block 840. In one or more implementations, the passive button exhibits function overloading behavior where different interactions with the passive button by the operator O can indicate different commands or functions. For example, in one or more implementations, pressing the passive button causes a toggle of the loss of actuation state and pressing and holding the passive button causes a slow entry or exit (and restoration of one or more physical parameters) to, or from, the loss of actuation state for the flexible elongate device 202. That is, when the flexible elongate device 202 is not in the loss of actuation state and the passive button is pressed, execution of Block 830 (i.e., to enter the loss of actuation state) is temporally quick (e.g., within 0.1 seconds). In contrast, when the flexible elongate device 202 is not in the loss of actuation state and the passive button is pressed and held (e.g., for 5 seconds), execution of Block 830 is temporally slow such that the flexible elongate device 202 slowly enters the loss of actuation state or slowly experiences a reduction in stiffness (e.g., over a period of 5 seconds). Continuing with this example, when the flexible elongate device 202 is in the loss of actuation state and the passive button is pressed, execution of Block 840 (i.e., to exit the loss of actuation state) is temporally quick (e.g., within 0.1 seconds). In one or more implementations, pressing the passive button while the flexible elongate device 202 is in the loss of actuation state causes the flexible elongate device 202 to exit the loss of actuation state (Block 840) and pauses the processes of FIG. 8 until additional input is received from the operator O, e.g., a confirmation or selection to execute the restoration process of Blocks 850 and 860. In contrast, when the flexible elongate device 202 is in the loss of actuation state and the passive button is pressed and held, the flexible elongate device 202 is caused to exit the loss of actuation state (Block 840) and the restoration process (Blocks 850 and 860) is performed while the passive button is held. In this case, if the operator O releases the passive button before the restoration process is completed, the restoration process can be paused and/or aborted. Other methods for function overloading the passive button can be implanted without limitation. For example, the flexible elongate device 202 can be placed in the loss of actuation state while the passive button is pressed and held and exit from the loss of actuation state-including a restoration of the first physical parameter (e.g., over a period of time)—if the passive button is released.

While the preceding paragraph describes an example where the master assembly 106 of the medical system 100 includes a passive button for receiving user input relating to the loss of actuation state for the flexible elongate device 202, other methods or techniques for receiving user input can be used, including to use of other types of input devices. For example, in one or more implementations, the display system 110 displays an interactive menu and/or graphical user interface to enter the loss of actuation state, exit the loss of actuation state, and command the restoration of one or more physical parameters for the flexible elongate device 202. Use of the menu and/or graphical user interface can be realized using the master assembly 106 or the display system 110 itself (e.g., display system includes a touch screen).

In one or more embodiments, more than one value or state for the first physical parameter can be stored (e.g., Block 710, Block 820) forming a history of values or states for the first physical parameter (e.g., organized or ordered according to a time of acquisition). In some implementations, restoration of the first physical parameter includes a selection, by a user, of a recorded value or state to be restored.

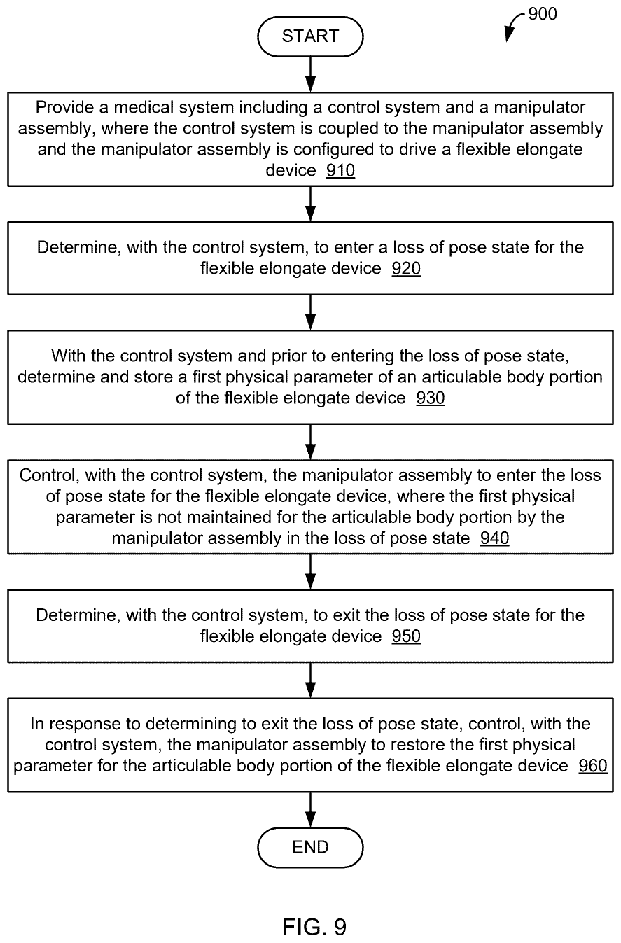

FIG. 9 depicts a method 900 for controlling a manipulator assembly to restore a first physical parameter to a flexible elongate device that has entered and exited from a loss of actuation state. Step 910 dictates that the method 900 is implemented using a medical system that includes a control system and a manipulator assembly. That is, in Step 910 a medical system including a control system and a manipulator assembly is provided. The control system is coupled to the manipulator assembly. Further, the manipulator assembly is configured to drive, or control, a flexible elongate device.

In Step 920, with the control system, it is determined that the flexible elongate device enters the loss of actuation state. In one or more implementations, the flexible elongate device is determined to enter the loss of actuation state in response to user input. In other embodiments, the flexible elongate device is determined to enter the loss of actuation state due to fault reaction logic of the medical system or the occurrence of an event-based inactive. Entry of the flexible elongate device into the loss of actuation state is caused by active control of the actuators, which is different from changes of shape and/or pose that are caused only by external force (e.g., prolapse, deflections, etc.).

In Step 930, prior to entering the loss of actuation state (as determined according to Step 920), the control system is used to determine and store a first physical parameter of an articulable body portion of the flexible elongate device. In one or more embodiments, the distal portion of the flexible elongate device is articulable and can be pointed in different directions and/or moved to different positions. An example of the first physical parameter is a pointing direction. In some implementations the pointing direction can be described as a state, e.g., the state of pointing at a specified target. Another example of the first physical parameter is a position of the distal portion or distal end of the flexible elongate device. Other non-limiting examples of the first physical parameter include a bend angle of the distal portion, a distance from a target, and apposition (or a state of apposition). The first physical parameter can include more than one descriptions or data elements. For example, a pointing direction can include one or more vectors used to define the direction and location of an axis that represents the pointing direction as well as the position of a target or other object or point that is intersected by the axis. The first physical parameter can be determined using one or more of a shape sensor, a position sensor, a contact force sensor, and intra-operative imaging. For example, in one or more embodiments, the flexible elongate device includes, or is associated with (e.g., as part of medical tool), a shape sensor such that the shape—or set of poses along a length of the flexible elongate device—is known or can be determined (e.g., using the control system). In these embodiments, the shape of the flexible elongate device is used to determine the first physical parameter. In some implementations, the shape of the flexible elongate device is determined, or validated, in view of other available information such as intra-operative images and a registration of the flexible elongate device to visualized and/or modelled patient anatomy. In general, the shape of the flexible elongate device, can be described as the sensed configuration of the flexible elongate device. Thus, in some implementations, the control system receives the sensed configuration of the flexible elongate device. The first physical parameter, or the data structure or computational object that describes the first physical parameter, is recorded in a memory of the medical system (e.g., memory of the control system).

In Step 940, the control system is used to control the manipulator assembly to enter the loss of actuation state for the flexible elongate device. Generally, the loss of actuation state is characterized by a reduction in stiffness and/or no specified articulation or shape for the flexible elongate device. That is, in the loss of actuation state, the flexible elongate device may be not controlled to any specific pose or shape and, in general, may be allowed to articulate to an unspecified shape based on the physical stiffness properties of the flexible elongate device. Thus, in the loss of actuation state, a specified pose, apposition, or shape (or a controlled or actuated pose, apposition, or shape, or, more simply, the pose or shape) of the flexible elongate device is not necessarily maintained. Entry to the loss of actuation state for the flexible elongate device is caused by active control of the actuators, which is different from changes of shape, apposition, and/or pose that are caused only by external force (e.g., prolapse, deflections, etc.). That is, the loss of actuation state is distinguished from changes to the articulation and/or shape of the flexible elongate device that are caused only by external force. In the loss of actuation state, the first physical parameter for the articulable body portion of the flexible elongate device is actively not maintained.

In Step 950, with the control system, it is determined to exit the loss of actuation state for the flexible elongate device. Exiting the loss of actuation state can be characterized by an ability to control or steer the flexible elongate device, e.g., though articulation of the articulable body portion. Exit from the loss of actuation state can include returning, or placing, the flexible elongate device to a hold state or following state. In one or more implementations, the flexible elongate device is determined to exit the loss of actuation state in response to a user input. In other embodiments, the determination to exit the loss of actuation state is made in response to fault reaction logic (e.g., due to the resolution of a fault) or an observed event (e.g., a reversal of an event-based inactive).

In Step 960, in response to determining to exit the loss of actuation state, the control system is used to control the manipulator assembly to restore the first physical parameter for the articulable body portion of the flexible elongate device. Restoration of the first physical parameter for the articulable body portion of the flexible elongate device includes articulation of the articulable body portion such that the first physical parameter is the same (or substantially equal to) the stored first physical parameter (see Step 930). In greater detail, the first physical parameter can be represented as a value or state (e.g., a vector representing the position of the distal end of the flexible elongate device, a state of pointing at a specified target, apposition, etc.). In Step 930 the value or state for the first physical parameter is stored. In exiting the loss of actuation state, the first physical parameter may have a different value or state than that stored (e.g., the position of the distal end of the flexible elongate device changed while in the loss of actuation state). Thus, restoring the first physical parameter includes controlling the articulable body portion of the flexible elongate device to move so that the value or state of the first physical parameter matches the stored value or parameter. Notably, restoration of the first physical parameter does not always require that the flexible elongate device have the same shape as when the first physical parameter was stored prior to the flexible elongate device entering the loss of actuation state. For example, a pointing direction can be restored to the articulable body portion of the flexible elongate device while other portions (e.g., the portion of the flexible elongate device proximal to the articulable body portion) have a different pose than before the flexible elongate device entered the loss of actuation state. Further, in the case of the pointing direction, even the articulable body portion may have a different shape or pose as long as the restored pointing direction is such that the articulable body portion is pointed or directed at a stored position, e.g., the position of a target.

In one or more implementations, the restoration of the first physical parameter includes the determination of a trajectory for the articulable body portion of the flexible elongate device. The trajectory can be determined, for example, to reduce a difference between the current value or state of the first physical parameter and the recorded value or state. Further, in some implementations, movement of the articulable body portion of the flexible elongate device to restore the first physical parameter is monitored. The movement can be monitored using one or more of a shape sensor, a position sensor, a contact force sensor, and intra-operative imaging (including one or more of internally and externally acquired image data). In one or more implementations, an error is determined between the monitored movement and the determined trajectory. In instances where the error exceeds a threshold, control of the manipulator assembly to restore the first physical parameter can be aborted. Examples of errors include a spatial distance between a position of the articulable body portion of the flexible elongate device and the trajectory during the monitored movement and a determined torque of an actuator that actuates the articulable body portion of the flexible elongate device. Thus, examples of thresholds used to evaluate the severity of the errors and/or induce a response in the medical system, e.g., abortion of the restoration process, include a distance threshold and a torque threshold. Other scenarios can cause abortion of control of the manipulator assembly to restore the first physical parameter. These scenarios include receiving a user input to abort the restoration process and receiving a user input to assume control of the flexible elongate device or portion thereof. For example, while the control system is controlling the manipulator assembly to restore the first physical parameter for the articulable body portion of the flexible elongate device, the operator may use the master assembly to steer the flexible elongate device. In this case, the restoration process is aborted and the control system controls the manipulator assembly to steer the flexible elongate device according to the input of the operator received at the master assembly.

In one or more embodiments, movement or control of the articulable body portion of the flexible elongate device to restore the first physical parameter is subject to, or limited by, one or more constraints. For example, the movement can be limited according to a given amplitude limit where an amplitude, such as a Euclidean distance or L2-norm, is determined for the movement of the articulable body portion and the movement is restricted to, or aborted, if the amplitude exceeds the amplitude limit. As another example, and as described above with respect to the determination of an error, one or more actuators used to actuate the articulable body portion of the flexible elongate device can be limited to one or more given torque limits. Further, a velocity or speed can be determined for the movement of the articulable body portion of the flexible elongate device and a speed limit can be applied to the movement. In some embodiments, a contact force limit can be applied. This may be applicable, for example, in configurations that include a contact force sensor, e.g., to sense a force at the distal end or tip of the flexible elongate device. This may be useful, for example, to sense a contact force between the tip and the surrounding airway.

In one or more embodiments, the restoration process is restricted based on a state of the medical system and/or the state of a procedure performed with the medical system. The state of the medical system, the state of a procedure performed by the medical system, or both, can be described according to an operation context. An example of an operation context includes the state of a biopsy needle conveyed by the flexible elongate device or the relative positions of the distal ends of the biopsy needle and flexible elongate device. In this example, the biopsy needle can either been extended (or protruding) or non-extended (or enclosed) relative to the flexible elongate device. In one or more implementations, the restoration process is not permitted or aborted if the a biopsy needle is extended. In general, the control system determines an operation context and control of the manipulator assembly to restore the first physical parameter for the articulable body portion of the flexible elongate device is allowed or restricted based on the determined operation context. For example, in one or more implementations, control of the manipulator assembly to restore the first physical parameter for the articulable body portion of the flexible elongate device is allowed when the operation context includes a non-extended biopsy needle.

An example medical system and its use in view of the method 900 of FIG. 9 is given as follows. In this example, the medical system is said to include a control system, a manipulator assembly, and a master assembly. The control system is coupled to the manipulator assembly and the master assembly. The manipulator assembly is configured to drive a flexible elongate device. The master assembly can be used, at least in part, to receive user input (e.g., from an operator), where the user input is processed by the control system and used to control the manipulator assembly to drive the flexible elongate device. For example, articulation of an articulable body portion of the flexible elongate device can be commanded or controlled by an operator using the master assembly. Further, in this example, the flexible elongate device includes a shape sensor used to determine a shape or sensed configuration for the flexible elongate device (or, at least an articulable body portion of the flexible elongate device) and the master assembly includes a passive button.

When the flexible elongate device is not in the loss of actuation state, pressing the passive button causes: 1) the control system to store one or more physical parameters relating to the articulable body portion of the flexible elongate device; and 2) the flexible elongate device (and its articulable body portion) to enter the loss of actuation state. When the flexible elongate device is not in the loss of actuation state, pressing and holding the passive button causes: 1) the control system to store one or more physical parameters relating to the articulable body portion of the flexible elongate device; and 2) the flexible elongate device to enter the loss of actuation state over a predefined period (e.g., 5 seconds). In greater detail, storing the one or more physical parameters includes storing a value or state (e.g., a vector, a data structure, etc.) that describe a physical parameter of the articulable body portion of the flexible elongate device. Examples of physical parameters for the articulable body portion of the flexible elongate device include a position and/or orientation, a pointing direction (which can include, e.g., a point or object that the articulable body portion is pointed at), a bend angle, a pose, an apposition (or apposition state), a distance to a target or other reference structure or datum.

When the flexible elongate device is in the loss of actuation state, pressing the passive button causes the flexible elongate device to exit the loss of actuation state. When the flexible elongate device is in the loss of actuation state, pressing and holding the passive button causes the flexible elongate devices to exit the loss of actuation state and automatically restore the one or more physical parameters to the articulable body portion of the flexible elongate device. In this scenario, the passive button is held until the one or more physical parameters are restored, an interrupt is received by the control system, or the process is otherwise aborted by the user, e.g., by no longer holding the passive button or assuming control of the flexible elongate device using the master assembly. Restoration of the one or more physical parameters includes controlling, with the control system, the manipulator assembly to articulate or move the articulable portion of the flexible elongate device such that a difference between the current value or state and the stored value or state for the one or more physical parameters is reduced.

Continuing with this example, the medical system is used to perform a medical procedure including the insertion of the flexible elongate device into the anatomy of a patient, e.g., through a natural orifice of the patient. The master assembly is used by an operator to navigate the flexible elongate device to a target region in the patient anatomy containing a target for biopsy. After navigating to the target region, the master assembly is further used by the operator to localize the distal portion of the flexible elongate device including aiming the distal end of the flexible elongate device at the target, where the distal portion is said to be the articulable body portion of the flexible elongate device. To facilitate entry of a medical tool including a biopsy needle into the flexible elongate device, the operator presses the passive button. In this example, the one or more physical parameters includes a first physical parameter of a pointing direction. Thus, in pressing the passive button, and prior to entering the loss of actuation state for the flexible elongate device, the control system determines and stores the pointing direction of the articulable body portion (or distal portion) of the flexible elongate device. For ease of reference, the stored value or state for the pointing direction can be referred to as the first pointing direction. The first pointing direction includes an orientation of an axis that is colinear with the articulable body portion of the flexible elongate device and a distance from the distal end of the flexible elongate device. Thus, the pointing direction describes, or can be used to define, a position of a point. The position of the point is said to be at the target. Then, the flexible elongate device is caused to enter the loss of actuation state characterized, at least in part, by a reduction in stiffness. While in the loss of actuation state, the operator (or an assistant to the operator) inserts the medical tool including the biopsy needle into the flexible elongate device. As a result of the flexible elongate device being in the loss of actuation state and/or the insertion of the medical tool, the pointing direction of the articulable body portion of the flexible elongate device changes and is said to be at a second pointing direction, where the second pointing direction is not the same as the first pointing direction. That is, the articulable body portion is not pointed at the target.

With the medical tool including the biopsy needle inserted into the flexible elongate device, the operator presses and holds the passive button. In response to the operator pressing and holding the passive button, the control system controls the manipulator assembly to restore the pointing direction to the articulable body portion of the flexible elongate device. Restoration of the pointing direction includes determining a movement or articulation of the articulable body portion of the flexible elongate device such that the articulable body portion of the flexible elongate device moves from the second pointing direction to a pointing direction (e.g., first pointing direction or other) such that the axis colinear with the articulable body portion intersects the stored position of the target and the distance of the distal end of the flexible elongate device is within an acceptable distance from the target.

Notably, restoration does not require that the shape of the flexible elongate device be the same as it was prior to entering the loss of actuation state. In general, the one or more physical parameters (such as pointing direction) can be restored to the articulable body portion of the flexible elongate device without need for all portions of the flexible elongate device (including the articulable body portion) to be restored to their previous poses. The movement or articulation of the articulable body portion of the flexible elongate device is determined using the sensed configuration or shape of the flexible elongate device. In some scenarios, the movement is further monitored using the sensed configuration or shape of the flexible elongate device. The control system controls the manipulator assembly to move the articulable body portion of the flexible elongate device from the second pointing direction to the first (or stored) pointing direction until the articulable body portion of the flexible elongate device is at the first pointing direction (where the first pointing direction can indicate that the distal portion of the flexible elongate device pointed at the target). In some scenarios, the restoration process can be aborted, for example, because the operator releases (or stops pressing) the passive button or an interrupt (e.g., torque limit, distance threshold, etc.) is received by the control system. Further, in response to the operator pressing and holding the passive button, the control system can determine an operation context including a determination of whether the biopsy needle is extended or non-extended. The restoration process is allowed when the operation context is that the biopsy needle is not extended.

In the present example, it is said that the operation context allows the restoration process to occur, no interrupts (e.g., torque limits) are received during the restoration process, and the passive button is held long enough for the restoration process to be completed. Thus, in this example, the operator aimed the distal end of the flexible elongate device at a target, caused the flexible elongate device to enter the loss of actuation state (by pressing the passive button) wherein the distal end of the flexible elongate device was not maintained to be aimed at the target, inserted a biopsy needle, and then caused the flexible elongate device to exit the loss of actuation state and return to being aimed at the target (by pressing and holding the passive button).

While the preceding example used pointing direction as the physical parameter, use of other physical parameters as described are equally applicable (e.g., apposition-related parameter(s)). Additionally, while not discussed in the preceding example, the control system can also inform the operator that the restoration process is complete or if an error has occurred using one or more alerts such as an audiovisual alert and/or displayed message. Further, while this example used a passive button other examples can be constructed where the flexible elongate device is caused to enter the loss of actuation state due to fault reaction logic or an event-based inactive and, in response to exiting the loss of actuation state (e.g., by resolving a fault), one or more physical parameters of the articulable body portion of the flexible elongate device can be restored-either automatically or through a user prompt.

In summary, the instant disclosure relates to medical systems and methods for operating such medical systems. Operating a medical system (such as medical systems that use flexible elongate devices (e.g., catheters or endoscopes)) may involve performing a medical operation (e.g., a biopsy) at a target site using a tool (e.g., a biopsy needle) inserted through the flexible elongate device.

Performing the medical operation may involve navigation, localization, and/or apposition tasks of the flexible elongate device. Navigation may involve significant movement along an insertion degree of freedom, in addition to movement along the articulation degrees of freedom, e.g., to follow a passage (such as an airway or other anatomical passageway). Localization includes a targeting operation and other such pre-targeting movements performed local to the target site, where the targeting operation includes aiming the flexible elongate device towards the target site. Apposition may include ensuring that a portion of the flexible elongate device is in contact with an adjacent anatomical structure.

Use of the flexible elongate device, e.g., in navigation and localization tasks including a targeting operation, involves articulation of one or more articulable portions of the flexible elongate device. For various reasons, during operation of the medical system, a flexible elongate device may enter the loss of actuation state. In the loss of actuation state, the shape of the flexible elongate device, and thus one or more physical parameters of an articulable body portion of the flexible elongate device, is actively not maintained.

Embodiments of the instant disclosure relate to automatically restoring one or more physical parameters of the flexible elongate device to a previous value or state prior to entering the loss of actuation state. As an example, the position and orientation of a distal end of the flexible elongate device can be referred to as a distal pose. During operation of the medical system, the flexible elongate device may be adjusted such that the distal pose is at a first distal pose where the distal end is directed at the target site. Upon entering, or during, the loss of actuation state, the shape of the flexible elongate device is actively not maintained resulting in the distal end of the flexible elongate device no longer being directed at the target site. That is, in response to the loss of actuation state, the distal pose is at a second distal pose. While no longer having the loss of actuation state, the distal pose is restored from the second distal pose to the first distal pose.

The restoration of the distal pose to the first distal pose can be enacted in a variety of ways, including: automatically articulating the flexible elongate device to the first distal pose in response to exiting the loss of actuation state (can be supervised and aborted by the user); and receiving an input command from a user to restore the distal end of the flexible elongate device to the first distal pose, e.g., with an indication that the loss of actuation state can be exited. User inputs can include pressing a button, pressing and holding a button, and selecting or enabling the restoration behavior using a graphical user interface (e.g., including menu items). The graphical user interface can be provided as a touchscreen.

The flexible elongate device may be considered to be in, or enter, the loss of actuation state in response to one or more of the following: a detected fault of the medical system such as an inactive actuator associated with control of the flexible elongate device and a loss of signals indicative of, or used to calculate the shape, of the flexible elongate device (i.e., fault reaction logic); one or more event-based inactives (e.g., a brake of the manipulator assembly being “off”); and a user-initiated mode such as a passive mode (e.g., the user may desire that the flexible elongate device be passive or in the loss of actuation state to more easily insert/retract a medical tool in the flexible elongate device). It is emphasized that entering the loss of actuation state is induced by the medical system and not an external force such as a force applied from the surroundings of the flexible elongate device (e.g., patient motion) or tool motion (e.g., insertion or retraction) of a medical tool conveyed by the flexible elongate device. In general, the loss of actuation state is commanded by the medical system (e.g., via control of actuators) in response to various conditions as described above (e.g., loss of signal indicative of the shape of the flexible elongate device).

While not in the loss of actuation state, the one or more physical parameters of the flexible elongate device is determined and stored by the medical system. A history of the one or more physical parameters can be stored (e.g., distal poses or positions recorded along with a timestamp). In some implementations, the medical system continually acquires or updates the one or more physical parameters. In other implementations, the one or more physical parameters is determined and stored according to a user input (e.g., “checkpointing”). Upon entering, or during, the loss of actuation state, the one or more physical parameters is actively not maintained and the articulable body portion of the flexible elongate device is said to be at a different, or second, value or state for the one or more physical parameters. In response to exiting the loss of actuation state, the articulable body portion of the flexible elongate device is restored from the second value or state for the one or more physical parameters to the stored, or first, value or state for the one or more physical parameters. In some implementations, the first value or state for the one or more physical parameters used for restoration is selected by a user from one or more stored values or states (e.g., a history). Notably, restoration of the one or more physical parameters does not necessarily require restoration of the shape of the flexible elongate device, in whole or in portion.

In one or more embodiments, the flexible elongate device includes a shape sensor to detect the shape of the flexible elongate device. The shape sensor is used to determine the one or more physical parameters for the articulable body portion of the flexible elongate device (e.g., a position of the distal end, a pointing direction, a distance from a target, etc.). In one or more embodiments, in response to exiting the loss of actuation state, the shape sensor is used to restore the one or more physical parameters from the second value or state to the first, or stored, value or state.

In one or more embodiments, a shape of the flexible elongate device is determined using one or more imaging devices. In some embodiments, a 3D scan is performed, and the position of the flexible elongate is identified in the resulting 3D image. For example, the 3D scan can be a cone beam computed tomography (CBCT) or other 3D imaging modality. In some embodiments a 2D scan is obtained using fluoroscopy imaging. Alternatively, 2.5D scans may be obtained using tomosynthesis imaging. One or more scans of the imaging device(s) are used to determine the shape of the flexible elongate device including the value or state for the one or more physical parameters. In one or more embodiments, in response to exiting the loss of actuation state the one or more scans are used to restore the one or more physical parameters for the articulable body portion of the flexible elongate device.

In some scenarios, automatic restoration of the one or more physical parameters may not be possible. For example, during the automatic restoration of the one or more physical parameters, an observed or estimated torque of an actuator may exceed a threshold. As another example, restoration of the one or more physical parameters can include a movement of the articulable body portion of the flexible elongate device and an accumulated error of the expected trajectory may exceed a threshold. In cases where automatic restoration is not possible, or where the movement of the articulable body portion of the flexible elongate device is determined to satisfy a stop condition (e.g., exceeding a threshold), the restoration can be paused or aborted. Pause or abortion of automatic restoration can further include providing an alert (e.g., audiovisual alert) to a user and reverting to manual control of the flexible elongate device by the user. In one or more embodiments, a user may supervise and pause or abort an automatic restoration, e.g., though a user input (e.g., depression of a button or input command to control the flexible elongate device).

In one or more embodiments, a set of operation contexts must be satisfied to enable automatic restoration of the one or more physical parameters for the articulable body portion of the flexible elongate device. An example operation context is that an inserted medical tool does not extend from the distal end of the flexible elongate device. Such a condition can be determined and checked by the medical system with a knowledge of the length of the flexible elongate device (or inserted length) and an insertion length of the medical tool.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed is:

1. A medical system comprising:
a manipulator assembly configured to drive a flexible elongate device; and
a control system coupled to the manipulator assembly, the control system configured to:
determine to enter a loss of actuation state for the flexible elongate device, the loss of actuation state comprising one of a loss of pose state or a loss of apposition state;
prior to entering the loss of actuation state, determine and store a first physical parameter of an articulable body portion of the flexible elongate device;
control the manipulator assembly to enter the loss of actuation state for the flexible elongate device, wherein the first physical parameter is not maintained for the articulable body portion by the manipulator assembly in the loss of actuation state;
determine to exit the loss of actuation state for the flexible elongate device; and
in response to determining to exit the loss of actuation state, control the manipulator assembly to restore the first physical parameter for the articulable body portion of the flexible elongate device.

2. The medical system of claim 1, wherein the first physical parameter comprises at least one selected from a group consisting of:
a contact force as sensed by a force sensor comprised by the flexible elongate device,
a confirmed apposition of a portion of the flexible elongate device,
a pose,
a pointing direction, and
at least one of an orientation and a position.

3. The medical system of claim 1, wherein controlling the manipulator assembly to enter the loss of actuation state for the flexible elongate device comprises controlling the manipulator assembly to decrease a stiffness of the articulable body portion of the flexible elongate device.

4. The medical system of claim 1, wherein the manipulator assembly is controlled to restore the first physical parameter in response to determining to exit the loss of actuation state and receiving a user input to restore the first physical parameter for the articulable body portion of the flexible elongate device.

5. The medical system of claim 1, wherein the control system is further configured to receive a user input to store the first physical parameter, wherein the first physical parameter is determined and stored in response to receiving the user input.

6. The medical system of claim 1, wherein the control system is further configured to receive a first user input to enter the loss of actuation state, wherein the determination to enter the loss of actuation state is made in response to receiving the first user input.

7. The medical system of claim 6, wherein the control system is further configured to receive a second user input to exit the loss of actuation state.

8. The medical system of claim 1, wherein the determination to enter the loss of actuation state is based on fault reaction logic of the medical system.

9. The medical system of claim 8, wherein the fault reaction logic comprises at least one fault selected from the group consisting of:
a loss of power or an off state of one or more actuators coupled to the flexible elongate device; and
a signal fault in a signal that indicates a shape of the flexible elongate device.

10. The medical system of claim 1, wherein control of the manipulator assembly to restore the first physical parameter is aborted in response to a user input.

11. The medical system of claim 10, wherein abortion to restore the first physical parameter for the articulable body portion of the flexible elongate device comprises pausing control of the manipulator assembly.

12. The medical system of claim 10, wherein the control system is further configured to provide an alert to a user in response to aborting the restoration of the first physical parameter for the articulable body portion of the flexible elongate device.

13. The medical system of claim 1, wherein in the control system is further configured to:
determine a trajectory of the articulable body portion of the flexible elongate device for restoring the first physical parameter;
monitor a movement of the articulable body portion of the flexible elongate device to restore the first physical parameter;
determine an error between the monitored movement and the trajectory; and
abort the control of the manipulator assembly to restore the first physical parameter for the articulable body portion of the flexible elongate device in response to the error exceeding a threshold.

14. The medical system of claim 13, wherein the control system is further configured to:
receive a sensed configuration of the flexible elongate device,
wherein the movement is monitored using the sensed configuration.

15. The medical system of claim 13, wherein the control system is further configured to:
receive a three-dimensional (3D) image of the articulable body portion of the flexible elongate device,
wherein the movement is monitored using the 3D image.

16. The medical system of claim 15, further comprising an imaging device wherein the 3D image is generated using the imaging device.

17. The medical system of claim 15, wherein the 3D image is generated using cone beam computed tomography (CBCT).

18. The medical system of claim 13, wherein determining the error comprises determining a spatial distance between a position of the articulable body portion of the flexible elongate device and the trajectory during the monitored movement.

19. The medical system of claim 18, wherein the threshold is a distance threshold.

20. The medical system of claim 13, wherein determining the error comprises determining a torque of an actuator that actuates the articulable body portion of the flexible elongate device.

21. The medical system of claim 20, wherein the threshold is a torque threshold.

22. The medical system of claim 1, wherein controlling the manipulator assembly to restore the first physical parameter for the articulable body portion of the flexible elongate device comprises at least one selected from the group consisting of applying an amplitude limit, applying a torque limit, applying a contact force limit, and applying a speed limit to a movement of the articulable body portion.

23. The medical system of claim 1, wherein the control system is further configured to:
determine a first operation context, and based on the first operation context, allow the control of the manipulator assembly to restore the first physical parameter for the articulable body portion of the flexible elongate device.

24. The medical system of claim 23, wherein the first operation context comprises a non-extended biopsy needle.

25. The medical system of claim 1, wherein the restoration of the first physical parameter for the articulable body portion of the flexible elongate device comprises an articulation of the articulable body portion.

26. A method for controlling a medical system comprising a manipulator assembly configured to drive a flexible elongate device, the method performed by a control system of the medical system and comprising:
determining to enter a loss of actuation state for the flexible elongate device, the loss of actuation state comprising one of a loss of pose state or a loss of apposition state;
prior to entering the loss of actuation state, determining and storing a first physical parameter of an articulable body portion of the flexible elongate device;
controlling the manipulator assembly to enter the loss of actuation state for the flexible elongate device, wherein the first physical parameter is not maintained for the articulable body portion by the manipulator assembly in the loss of actuation state;
determining to exit the loss of actuation state for the flexible elongate device; and
in response to determining to exit the loss of actuation state, controlling the manipulator assembly to restore the first physical parameter for the articulable body portion of the flexible elongate device.

27. A non-transitory machine-readable medium comprising a plurality of machine-readable instructions executed by one or more processors associated with a medical system comprising a manipulator assembly configured to drive a flexible elongate device, the plurality of machine-readable instructions causing the one or more processors to perform a method comprising:
determining to enter a loss of actuation state for the flexible elongate device, the loss of actuation state comprising one of a loss of pose state or a loss of apposition state;
prior to entering the loss of actuation state, determining and storing a first physical parameter of an articulable body portion of the flexible elongate device;
controlling the manipulator assembly to enter the loss of actuation state for the flexible elongate device, wherein the first physical parameter is not maintained for the articulable body portion by the manipulator assembly in the loss of actuation state;
determining to exit the loss of actuation state for the flexible elongate device; and
in response to determining to exit the loss of actuation state, controlling the manipulator assembly to restore the first physical parameter for the articulable body portion of the flexible elongate device.
